(19) United States
(12) Patent Application Publication
(10) Pub. No.: US 2008/0067068 A1
(43) Pub. Date: Mar. 20, 2008

(54) DC-dielectrophoresis microfluidic apparatus, and applications of same

(75) Inventor: Dongqing Li, Antioch, TN (US)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: Vanderbilt University, Nashville, TN (US)

(21) Appl. No.: 11/523,782

(22) Filed: Sep. 19, 2006

(51) Int. Cl. C07K 1/26; G01N 27/00

(52) U.S. Cl. 204/451; 204/601

(57) ABSTRACT

The present invention relates to an apparatus and methods of separating particles or cells according to their sizes, wherein the size of each of the particles or cells is characterized by a corresponding diameter. In one embodiment, the method includes the steps of providing a microchannel structure having at least one channel that is defined by a first sidewall and a second, opposite sidewall and has an insulating protrusion formed on one of the first sidewall and the second, opposite sidewall, introducing a plurality of particles or cells in a liquid medium into the at least one channel, and generating a non-uniform electrical field in the at least one channel such that when the plurality of particles or cells passes by the insulating protrusion, the plurality of particles or cells each receives a dielectrophoretic force proportional to its diameters, thereby being separable according to their sizes. The method further has the step of collecting particles or cells after the separation of particles or cells.

### CROSS-REFERENCE TO RELATED PATENT APPLICATION

This application is related to a co-pending U.S. patent application entitled “Microfluidic Flow Cytometer and Applications of Same”, by Dongqing Li with Attorney Docket No. 14506-55548, filed Sep. 19, 2006, which has the same assignee as the present application and has been concurrently filed herewith. The applicant of that application is also applicant of this application. The disclosure of the above-identified co-pending application is incorporated in its entirety herein by reference.

Some references, which may include patents, patent applications and various publications, are cited and/or discussed in the description of this invention. The citation and/or discussion of such references is provided merely to clarify the description of the present invention and is not an admission that any such reference is “prior art” to the invention described herein. All references cited including those listed in the List of References and/or discussed in this specification are incorporated herein by reference in their entireties and to the same extent as if each reference was individually incorporated by reference.

### FIELD OF THE INVENTION

The present invention generally relates to microfluidics and in particular to DC-Dielectrophoresis microfluidic apparatus, and applications of same including separation of biological cells according to their sizes.

### BACKGROUND OF THE INVENTION

Microfluidics deals with the behavior, precise control and manipulation of microliter and nanoliter volume of fluids. It is a multidisciplinary field comprising physics, chemistry, engineering and biotechnology, with practical applications to the design of systems in which such a small volumes of fluids will be used. Microfluidics is used in the development of DNA chips, micro-propulsion, micro-thermal technologies, and lab-on-a chip technology.

Lab-on-a-chip devices are miniaturized biomedical laboratories on a credit card sized glass/plastic plate. These lab chips can duplicate the specialized functions of their room-sized counterparts in clinical diagnoses and tests. The advantages of these lab-on-a-chip devices include significantly reduced sample/reagent consumption, very short analysis time, high throughput and portability. Ideally, a lab-chip should be able to directly take a drop of whole blood and start the analysis. Currently, however, all lab-on-a-chip devices require purified DNA sample, because these devices do not have the capability to separate white blood cells from the whole blood to extract DNA. Thus the lab-on-a-chip devices still rely on conventional room-sized laboratories for blood sample pretreatment. This is a major limitation to the development and applications of lab-on-a-chip technology. Generally, a whole blood sample contains plasma, erythrocytes or red blood cells (RBC), leukocytes or white blood cells (WBC), and thrombocytes or platelets. Only 3% of the blood cells are WBC. The size of RBC is about 6 to 8 μm, the size of WBC ranges from 10 to 15 μm. In the conventional blood sample preparation, separating the WBC requires using centrifuge, which is not suitable for lab-on-a-chip devices.

Very few alternative cell separation methods are available. Magnetic cell separation (MACS) method was used to sort out cancer cells. However, this method requires using nanometer-sized magnetic beads coated with specific antigens/antibodies that attract certain cells. After the desired cells are attached to the magnetic beads, an external magnetic field is applied to separate these cells from the rest. This method may not work with normal blood cells, and is expansive (the cost of the nano magnetic beads). Furthermore, the magnetic beads attached to cells have to be separated from the cells for the subsequent analyses and DNA amplification (polymerase chain reaction (PCR)) processes.

Dielectrophoretic field-flow-fractionation (DEP-FFF) was applied to cancer cell separation. Cell separations were achieved in a thin chamber equipped with a microfabricated, interdigitated electrode array on its bottom wall that was energized with AC electric signals. Cells were levitated by the balance between dielectrophoresis (DEP) and sedimentation forces to different equilibrium heights and were transported at differing velocities and thereby separated when a velocity profile was established in the chamber. This method requires complicated, microfabricated, interdigitated electrode array on the chamber wall, and hence the cost of the device is high and the electronic operation control is sophisticated. Furthermore, pressure-driven flow must be used in this method to generate a parabolic velocity profile in the chamber; the cell separation efficiency is therefore dependent on the flow control as well. Additionally, this requires relatively large, external pump, tubing and valves and thus limits the portability of the device.

Recently the DC-DEP was employed for particle trapping and concentration in Microsystems. An insulator based DEP device was developed with an array of insulating rods in a microchannel, DEP trapping of 200 nm polystyrene particles was realized. Selective trapping of polystyrene particles, live E. coli, and dead E. coli in arrays of insulating posts using DC electric fields was demonstrated. However, no one has shown the separation of particles or cells by size in DC electrokinetic flow by DC-DEP.

Technologies that can separate a small volume of biological cells according to the size have particularly important applications in clinical detection and analysis of circulating tumor cells. While cancer has being diagnosed at increasingly earlier stages, most patients continue to develop into metastatic disease. When cancer spreads, or metastasizes, it travels through either the lymph channels or the bloodstream. There are urgent needs to develop methods that can efficiently recognize invasive tumor cells appearing in the peripheral blood circulation and in other body fluids. Finding the few circulating tumor cells among millions of normal cells will enable not only early diagnosis of cancer, but also biomarker studies. The molecular and genetic abnormalities within these exfoliated cells could be used to detect and identify precancerous lesions or very early stage cancer if highly sensitive technologies were clinically available to identify the few abnormal cells among millions of normal cells. During the early stages of cancer development, there is a window of opportunity to detect precancerous cells with genetic or molecular biomarkers that identify and characterize their progression towards cancer. Finding molecular and genetic biomarkers of malignancy is particularly important in detecting the emergence of precancerous cell populations and is considered by National Cancer Institute (NCI) to be an “Extraordinary Opportunity.”

In order to detect and analyze precancerous and cancerous cells in biologic fluids, a variety of approaches are available. However, all of these approaches require an enrichment of atypical epithelial cells through selective processing to concentrate the assay target of interest. The current tumor cell enrichment methods can be grouped into the following two broad categories: mechanical (e.g., centrifugation, cytospin, sucrose gradients); and antibody-based selection with mechanical separation (e.g., flow-assisted cell sorting (FACS), magnetic-assisted cell sorting (MACS)). All of these methods have good but not adequate sensitivity or specificity required for detecting precancerous cells in body fluids. Considering the fact that the concentration of these cells can be very low compared to other commonly present cell types, one needs to be able to isolate/separate one tumor cell out of 10,000 or one million normal cells. Therefore, it is highly desirable to develop novel and sensitive technologies for isolating the small numbers of exfoliated tumor cells in bio-fluids such as blood.

Women with advanced breast cancer who have a higher number of tumor cells circulating in their blood progress more rapidly and die sooner than women with fewer of these cells. Identifying the number of circulating tumor cells in patients with metastatic breast cancer, especially at the time of their first follow-up after starting new therapy, may provide an early, reliable indication of whether that therapy will be successful. A study used a technology called CellSearch™ that isolates and characterizes these cells. The CellSearch™ technique involves mixing a blood sample with iron particles coated with an antibody that attaches to epithelial cells like those found in breast tissue. The cells are further characterized with other antibodies that have been tagged with a fluorescent dye, so that the cancer cells can be easily distinguished and counted. Since epithelial cells are not typically found in blood, their presence suggests they are cancerous cells from the breast tissue.

Morphometric analyses of tumor cell lines derived from liver, prostate, breast, and cervix human carcinomas have confirmed the significantly larger cell size (20 μm and larger) of these carcinoma cells as compared to peripheral blood leukocytes (˜12 μm). Based on this fact, a filtering method called ISET was developed to isolate epithelial tumor cells by size. This study demonstrated isolation by size of circulating tumorous cells in patients with carcinoma. ISET uses a module of filtration (Biocom company, Les Ulis, France) and a polycarbonate Track-Etch-type membrane (Cyclotron Technology) with calibrated, 8-μm-diameter, cylindrical pores. Sample is filtered through a 0.6-cm-diameter surface area in the membrane. Ten milliters of diluted solution, corresponding to 1 ml of undiluted blood, was loaded on a well and filtered by gentle aspiration under vacuum (created by a vacuum pump). The membrane was then washed once by aspiration with phosphate-buffered saline (PBS), disassembled from the filtration module, and allowed to air dry and microscope examination. It was reported that this ISET method can detect a single, micropipetted tumor cell added to 1 ml of blood.

All these existing methods require cumbersome, multiple operation procedures, complicated instruments, and labeling (e.g., magnetic beads, antibodies, and dyes). These processes often cause cell losses and damage to the cells.

Therefore, a heretofore unaddressed need exists in the art to address the aforementioned deficiencies and inadequacies.

### SUMMARY OF THE INVENTION

In one aspect, the present invention relates to a microchannel structure that can be utilized to separate particles or cells in a liquid medium according to their sizes. In one embodiment, the microchannel structure includes:

a substrate having a first end, and an opposite, second end defining a body portion therebetween, wherein the body portion has a first surface and an opposite, second surface;

a first channel formed on the first surface of the substrate with a width, W₁, defined by a first sidewall and a second, opposite sidewall;

a second channel formed on the first surface of the substrate with a width, W₂, defined by a first sidewall and a second, opposite sidewall, wherein the second channel is in fluid communication with the first channel at a first at least three-way intersection; and

an insulating hurdle member having a top portion and protruding from the first sidewall of the first channel, wherein the top portion of the insulating hurdle member and the second sidewall of the first channel defines a width, W₁ₐ, therebetween, and wherein W₁ and W₁ₐ satisfy the relationship of W₁>W₁ₐ.

The top portion of the hurdle member has at least one corner with a corresponding angle α, wherein the angle α is in the range of 0 to 90°. The hurdle member can have a cross-section in a variety of geometric shapes. The hurdle member, in one embodiment, is substantially rectangular cross-sectionally, and the top portion of the hurdle member has two corners. The hurdle member, in another embodiment, is substantially triangular cross-sectionally, and the top portion of the hurdle member has one corner. The top portion of the hurdle member, in a further embodiment, has a surface characterized by a curvature. The hurdle member, in yet another embodiment, is in the form of an insulating liquid droplet, and the surface of the top portion of the hurdle member is at least partially spherical.

The microchannel structure further has a third channel formed on the first surface of the substrate with a width, W₃, defined by a first sidewall and a second, opposite sidewall, wherein the third channel is in fluid communication with the first channel at a second at least three-way intersection, wherein the third channel is formed on the first surface of the substrate such that the hurdle member is positioned between the first at least three-way intersection and the second at least three-way intersection, wherein the width, W₃, is same or different from at least one of the width, W₁, and the width, W₂. The width, W₁, can be same or different from width, W₂.

The substrate, in one embodiment, is formed with at least one insulating polymeric material. The insulating polymeric material can be PDMA. The hurdle member can be made from a material different from or substantially same as the at least one insulating polymeric material.

In another embodiment, the first channel is formed with a first portion with a width, W₁, and a second portion with a width, W_{1b}, which is defined by a first sidewall portion and a second sidewall portion, wherein the first sidewall portion is located between the top portion of the hurdle member and the first at least three-way intersection, and the second sidewall portion is located between the top portion of the hurdle member and the first at least three-way intersection, respectively, and wherein the width, W_{1b}, is varied at least for a portion along the first channel between the top portion of the hurdle member and the first at least at least three-way intersection, and wherein W_{1b}>W₁. The width W_{1b} of the second portion of the first channel proximate to the first at least three-way intersection is larger than the width W_{1b} of the second portion of the first channel proximate to the hurdle member.

Each of the first at least three-way intersection and the second at least three-way intersection can be an N-way intersection, where N is an integer no smaller than 3. For example, in one embodiment, the first at least three-way intersection is a three-way intersection that is substantially T-shaped. In another embodiment, the second at least three-way intersection is a three-way intersection that is substantially T-shaped. The first at least three-way intersection and the second at least three-way intersection can have other shapes, and can be same or different from each other.

The microchannel structure further has an insulating base member, wherein the insulating base member is bonded with the substrate to form a sealed microchannel structure. In one embodiment, the insulating base member is a glass plate.

In one embodiment, the microchannel structure further has a first well in fluid communication with the first channel at a first end of the first channel, a second well in fluid communication with the second channel at a first end of the second channel, a third well in fluid communication with the second channel at a second end of the second channel, which is apart from the first end of the second channel, and a fourth well in fluid communication with the third channel at a first end of the third channel, respectively.

In one embodiment, the microchannel structure further has a first electrode configured to be positioned in the first well and to be electrically connectable to a power source, a second electrode configured to be positioned in the second well and to be electrically connectable to a power source, a third electrode configured to be positioned in the third well and to be electrically connectable to a power source, and a fourth electrode configured to be positioned in the fourth well and to be electrically connectable to a power source, respectively.

A microfluidic chip can be formed with one or more microchannel structures as set forth above. The one or more microchannel structures can be arranged in an array. A device can be made with one or more such microfluidic chips.

In a further aspect, the present invention relates to a method of separating particles or cells according to their sizes, wherein the size of each of the particles or cells is characterized by a corresponding diameter. In one embodiment, the method includes the steps of:

providing a microchannel structure having a substrate having a first end, and an opposite, second end defining a body portion therebetween, wherein the body portion having a first surface and an opposite, second surface, a first channel formed on the first surface of the substrate with a width, W₁, defined by a first sidewall and a second, opposite sidewall, a second channel formed on the first surface of the substrate with a width, W₂, defined by a first sidewall and a second, opposite sidewall, wherein the second channel is in fluid communication with the first channel at a first at least three-way intersection, and an insulating hurdle member having a top portion and protruding from the first sidewall of the first channel, wherein the top portion of the insulating hurdle member and the second sidewall of the first channel defines a width, W₁ₐ, therebetween, and wherein W₁ and W₁ₐ satisfy the relationship of W₁>W₁ₐ;

introducing a plurality of particles or cells in a liquid medium into the microchannel structure; and

applying a direct current (DC) electrical field within the microchannel structure to generate a non-uniform electrical field at least around the insulating hurdle member and a first voltage difference along the first channel such that the plurality of particles or cells is driven by the direct current (DC) electrical field along the first channel and separated according to their diameters by a dielectrophoretic force corresponding to the non-uniform electrical field when the plurality of particles or cells passes by the insulating hurdle member.

In one embodiment, the step of applying a direct current (DC) electrical field further includes the step of generating a second voltage difference along the second channel such that at the first at least three-way intersection, a first group of the plurality of particles or cells moves to the second channel along a first direction, Y₁, and a second group of the plurality of particles or cells moves to the second channel along a second direction, Y₂, that is different from the first direction, respectively, wherein each of the first group of the plurality of particles or cells has a diameter that is larger than a predetermined diameter threshold, and each of the second group of the plurality of particles or cells has a diameter that is not larger than the predetermined diameter threshold.

The method further has the step of collecting particles or cells after the separation of particles or cells according to their sizes. The plurality of particles or cells can contain white blood cells and red blood cells. The plurality of particles or cells can also contain normal cells and tumor cells.

In one embodiment, the microchannel structure further has a third channel formed on the first surface of the substrate with a width, W₃, defined by a first sidewall and a second, opposite sidewall, wherein the third channel is in fluid communication with the first channel at a second at least three-way intersection, wherein the third channel is formed on the first surface of the substrate such that the hurdle member is positioned between the first at least three-way intersection and the second at least three-way intersection.

In another aspect, the present invention relates to a method of separating particles or cells according to their sizes, wherein the size of each of the particles or cells is characterized by a corresponding diameter. In one embodiment, the method includes the steps of providing a microchannel structure having at least one channel that is defined by a first sidewall and a second, opposite sidewall and has an insulating protrusion formed on one of the first sidewall and the second, opposite sidewall, introducing a plurality of particles or cells in a liquid medium into the at least one channel, and generating a non-uniform electrical field in the at least one channel such that when the plurality of particles or cells passes by the insulating protrusion, the plurality of particles or cells each receives a dielectrophoretic force proportional to its diameters, thereby being separable according to their sizes. The method further has the step of collecting particles or cells after the separation of particles or cells.

In yet another aspect, the present invention relates to an apparatus of separating particles or cells according to their sizes, wherein the size of each of the particles or cells is characterized by a corresponding diameter. In one embodiment, the apparatus has a microchannel structure having at least one channel that is defined by a first sidewall and a second, opposite sidewall and has an insulating protrusion formed on one of the first sidewall and the second, opposite sidewall, and means for generating a non-uniform electrical field in the at least one channel such that when the plurality of particles or cells passes by the insulating protrusion, the plurality of particles or cells each receives a dielectrophoretic force proportional to its diameters, thereby being separable according to their sizes. In another embodiment, the apparatus has a plurality of microchannel structures in an array.

In one embodiment, the means for generating a non-uniform electrical field includes a DC power source, a conducting liquid containable in the at least one channel and a plurality of electrodes configured to be electrically connectable to the DC power source and when connected to the DC power source, a non-uniform electrical field is generated at least in the at least one channel. The means for generating a non-uniform electrical field further has a voltage controller electrically coupled to the DC power source and the plurality of electrodes, wherein the voltage controller is capable of controlling the voltage output of each of the plurality of electrodes independently.

The apparatus further has means for receiving the plurality of particles or cells, and means for collecting the plurality of particles or cells after the separation of the plurality of particles or cells.

The particles or cells are normally provided in a liquid medium of interest, which may comprise a biological fluid of a living subject. The biological fluid includes blood or urine. The blood or urine comprises one or more types of particles or cells. The one or more types of cells are differentiable by their sizes, functions or a combination of them. The one or more types of cells may comprise red blood cells, white blood cells, CD4+ cells, and/or CD3+ cells. The one or more types of cells may be associated with a disease, which may be then detected and/or treated through the cells.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

FIG. 1 (A) shows contour of a DC electric field E and electric-field lines around an insulating hurdle in a microchannel. The darkness level indicates the magnitude of E. The x-direction is the channel length (the flow) direction, and the y-direction is the channel width direction. The coordinates are normalized by channel width h. (B) shows an enlarged view of a particle moving around the edge region of the hurdle. The darkness level indicates the magnitude of the electric field strength.

FIG. 2 schematically illustrates a microchannel structure according to one embodiment of the invention: (a) a perspective view; (b) a partial, perspective view of a microchannel structure formed on a substrate; and (c) a schematic, top view of (b).

FIG. 3 shows superimposed sequential images of separation of white blood cells in lysed blood solution by DC-DEP. The channel width is about 300 μm. The gap width between the channel and the hurdle is 40 μm. The applied voltages at different branches are indicated. (a) 10 μm threshold separation; and (b) 5 μm threshold separation.

FIG. 4 is a schematic drawing of a microchannel structure formed on a substrate according to one embodiment of the invention with a rectangular hurdle.

FIG. 5 shows: (a) a microchannel structure formed on a substrate according to one embodiment of the invention with a triangle hurdle; and (b) a microchannel structure of (a) but with a diverging channel section.

FIG. 6 shows a chip using a microchannel structure according to one embodiment of the invention: (a) a photo image of the chip; and (b) dimension of the chip and corresponding inner structure (the inset).

FIG. 7 shows separation of 5.7 and 15.7 μm particles at 500 V voltage level. Applied voltages at different electrodes: V_{A}=54 V, V_{B}=244 V, V_{C}=502 V, V_{D}=0 V, (a) superposed particle trajectories; and (b) comparison of the simulation results and the experimental data.

FIG. 8 shows separation of 5.7 and 15.7 μm particles at different voltage levels. Voltage level increases from 500 to 900 V. Applied voltages at different electrodes are specified in Table 1. (a) Superposed particle trajectories; and (b) comparison of the simulation results and the experimental data.

FIG. 9 schematically shows effects of branch voltage VA on the separation of 5.7 and 15.7 μm particles with V_{B}=244 V, V_{C}=502 V: (a) V_{A}<4 V; (b) 4 V<V_{A}<108 V; (c) V_{A}>108 V.

FIG. 10 shows visualization of the fluid flow (streamlines) in the microchannel. Applied voltages at different electrodes: V_{A}=54 V, V_{B}=244 V, V_{C}=502 V, V_{D}=0 V. (a) Experimental result; (b) simulation result.

FIG. 11 shows an example of superimposed sequential microscope images of the separation of 6 μm and 15 μm polystyrene particles by DC-DEP. The maximum electrical field in the channel is about 80V/cm.

FIG. 12 schematically shows a PDMS microfluidic chip for particle separation according to one embodiment of the invention, where oil from branch 4 forms a droplet in the main channel 1-3. Teflon tubing for oil delivery is press fitted in the PDMS lid of the oil reservoir.

FIG. 13 shows superimposed trajectories of the 5.7 μm and 15.7 μm particles separated under 400 V applied at reservoir 1 and 160 V at reservoir 2 of FIG. 12. The gap between the droplet and the channel wall was 46 μm wide.

FIG. 14 shows the effect of the applied voltage at reservoir 1 of FIG. 12 on the separation of 5.7 μm and 15.7 μm particles with the 46 mm wide gap. In all three cases the large and small particles were separated by approximately 240 μm.

FIG. 15 shows the effect of the gap width of (a) 95 μm and (b) 197 μm on (c) the separation of 5.7 μm and 15.7 μm particles under the applied 600 V at reservoir 1 of FIG. 12. The downstream separation of particles decreased with the increasing gap width.

FIG. 16 shows separation image of 1 μm and ˜5.7 μm particles at the gap width of 16 μm under the applied 200 V at reservoir 1 of FIG. 12.

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. Referring to the drawings, like numbers indicate like parts throughout the views. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which has no influence on the scope of the invention. Additionally, some terms used in this specification are more specifically defined below.

### DEFINITIONS

The terms used in this specification generally have their ordinary meanings in the art, within the context of the invention, and in the specific context where each term is used.

Certain terms that are used to describe the invention are discussed below, or elsewhere in the specification, to provide additional guidance to the practitioner in describing the apparatus and methods of the invention and how to make and use them. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to various embodiments given in this specification. Furthermore, subtitles may be used to help a reader of the specification to read through the specification, which the usage of subtitles, however, has no influence on the scope of the invention.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The term “lab-on-a-chip” or its acronym “LOC”, as used herein, refers to a device that has at least one microchannel structure, and that integrates multiple laboratory functions (processes) on a single chip of only millimeters to a few square centimeters in size. The LOC is capable of handling substantially small fluid volumes down to less than picoliters to perform desired biological and/or chemical analysis.

As used herein, the term “microchannel” refers to a channel structure having a cross-sectional dimension, e.g., a width, a depth or a diameter, in a microscale range from about 0.1 μm to about 1 mm. According to the present invention, the microchannels preferably have a cross-sectional dimension between about 0.1 μm and 500 μm, more preferably between about 0.1 μm and 300 μm. A device referred to as being microscale includes at least one structural element or feature having such a dimension.

As used herein, the term “microfluidics” refers to the science of designing, manufacturing, and formulating devices and processes that deal with volumes of fluid on the order of nanoliters (nl) or picoliters (pl). A microfluidic device has one or more channels with a cross-sectional dimension less than 1 mm. Common fluids used in microfluidic devices include whole blood samples, bacterial cell suspensions, protein or antibody solutions and various buffers. Applications for microfluidic devices include, but not limited to, capillary electrophoresis, isoelectric focusing, immunoassays, flow cytometry, sample injection of proteins for analysis via mass spectrometry, PCR (polymerase chain reaction) amplification, DNA (deoxyribonucleic acid) analysis, cell manipulation, cell separation, cell patterning and chemical gradient formation. Many of these applications have utility for clinical diagnostics.

As used herein, the term “electrokinetics” refers to the science of electrical charges in moving substances, such as water or blood, which studies particle motion that is the direct result of applied electric fields. Electrokinetics includes electroosmosis, electrophoresis, dielectrophoresis and electrorotation.

Electroosmosis, also called electroendosmosis, is the motion of polar liquid through a membrane or other porous structure (generally, along charged surfaces of any shape and also through non-macroporous materials which have ionic sites and allow for water uptake, the latter sometimes referred to as “chemical porosity”) under the influence of an applied electric field.

When a solid surface is in contact with an aqueous solution, electrostatic charge will be established at the surface. These surface charges in turn attract the counter ions in the liquid to the region close to the solid-liquid interface to form the electrical double layer. In the electrical double layer region, there are excess counter ions (net charge). If the solid surface is negatively charged, the counter ions are the positive ions. Such an electrical double layer field is responsible for two basic electrokinetic phenomena: electroosmosis and electrophoresis. When an external electrical field is applied tangentially to the solid surface, the excess counter ions will move under the influence of the applied electrical field, pulling the liquid with them and resulting in electroosmotic flow. The liquid movement is carried through to the rest of the liquid in the microchannel by the viscous effect. In most LOC applications, electroosmotic flow is preferred over pressure driven flow. One of the reasons is the plug-like velocity profile of electroosmotic flow. This means that fluid samples can be transported without dispersion caused by flow shear. Furthermore, pumping a liquid through a small microchannel requires applying a very large pressure difference depending on the flow rate. This is often impossible because of the limitations of the size and the mechanical strength of the microfluidic devices. Electroosmotic flow can generate the required flow rate in very small microchannels without any applied pressure difference cross the channel. Additionally, using electroosmotic flow to transport liquids in complicated microchannel networks does not require any external mechanical pump or moving parts, it can be easily realized by controlling the applied electrical fields via electrodes.

Electrophoresis is the motion of a charged particle relative to the surrounding liquid under an applied electrical field. In a microchannel, the net velocity of a charged particle is determined by the electroosmotic velocity of the liquid and the electrophoretic velocity of the particle. If the surface charge of the particle is not strong or the ionic concentration of the liquid (e.g., typical buffer solutions) is high, the particle will move with the liquid. Using electrical fields to manipulate and transport particles and biological cells in microchannels is particularly suitable for LOC applications.

It should be noted that the applied electrical field has negligible effects on the cells, other than generating the flow and the cell motion. This can be appreciated by comparing the applied electrical field strength with the electrical field strength of the cells' electrical double layer (EDL) field, i.e., the field around each cell generated by the natural surface electrostatic charge. The typical EDL field strength is 100 mV/10 nm=100,000 V/cm, while the applied electrical field ranges from 10 V/cm to 100 V/cm.

Dielectrophoresis or its acronym “DEP” refers to a phenomenon in which a force is exerted on a dielectric particle when it is subjected to a non-uniform electric field. This force does not require the particle to be charged. All particles exhibit dielectrophoretic activity in the presence of electric fields. However, the strength of the force depends strongly on the medium and particles' electrical properties, on the particles' shape and size, as well as on the frequency of the electric field. Consequently, fields of a particular frequency can manipulate particles with great selectivity. This has allowed, for example, the separation of cells or the orientation and manipulation of nanoparticles.

### OVERVIEW OF THE INVENTION

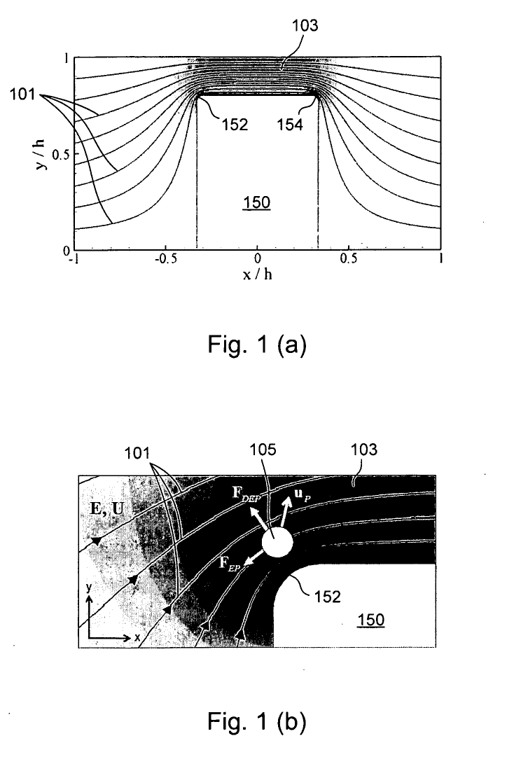

The description will be made as to the embodiments of the present invention in conjunction with the accompanying drawings of FIGS. 1-16. In accordance with the purposes of this invention, as embodied and broadly described herein, this invention, in one aspect, relates to a method and device for separating particles or cells of different sizes in a liquid medium according to their sizes.

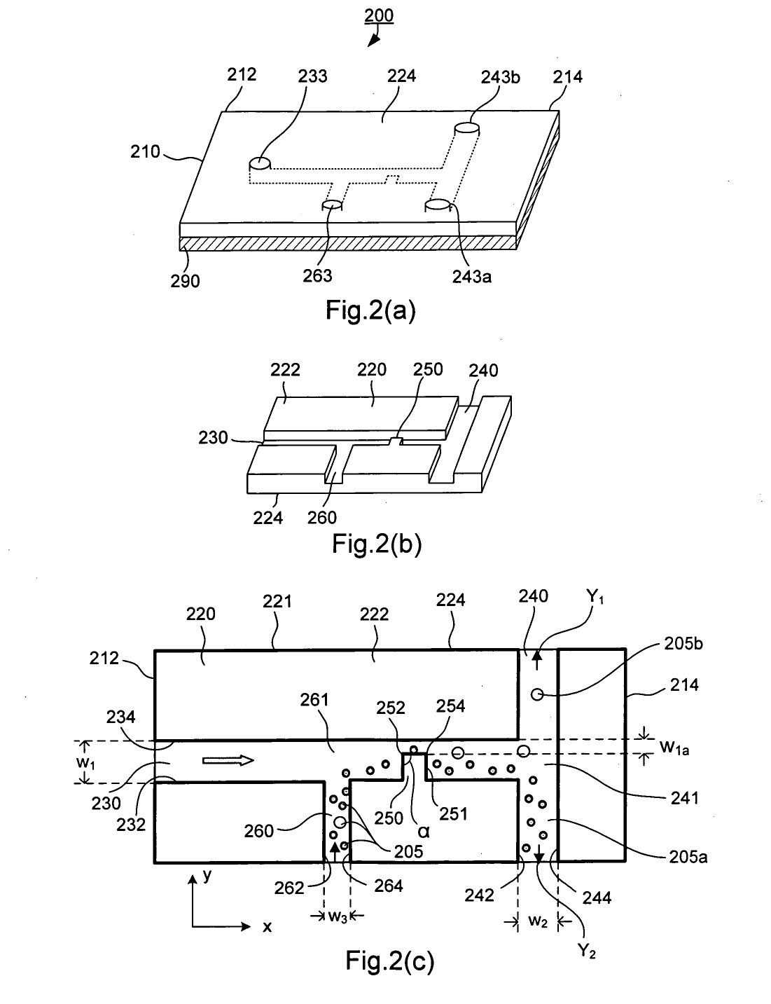

Referring first to FIGS. 2(a-c), a microchannel structure 200 according to one embodiment of the present invention is shown. The microchannel structure 200 has a substrate 210 that has a first end 212, and an opposite, second end 214 defining a body portion 220 therebetween, wherein the body portion 220 has a first surface 222 and an opposite, second surface 224. A first channel 230 is formed on the first surface 222 of the substrate 210 with a width, W₁, defined by a first sidewall 232 and a second, opposite sidewall 234. A second channel 240 is formed on the first surface 222 of the substrate 210 with a width, W₂, defined by a first sidewall 242 and a second, opposite sidewall 244, wherein the second channel 240 is in fluid communication with the first channel 230 at a first three-way intersection 241. And an insulating hurdle member 250, which has a top portion 251, protrudes from the first sidewall 232 of the first channel 230, wherein the top portion 251 of the insulating hurdle member 250 and the second sidewall 234 of the first channel 230 defines a width, W₁ₐ, therebetween, and wherein W₁ and W₁ₐ satisfy the relationship of W₁>W₁ₐ.

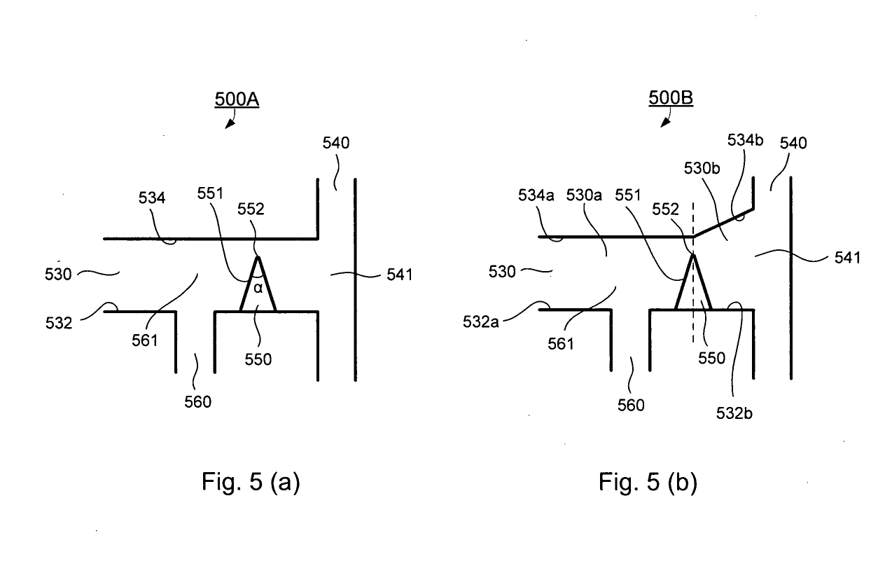

The hurdle member can be formed with different geometric shapes as well as different materials. As shown in FIG. 2, the hurdle member 250 is substantially rectangular cross-sectionally, and the top portion 251 of the hurdle member 250 has two corners 252, 254, each with a corresponding angle α, wherein the angle α is in the range of 0 to 90°. As shown in FIG. 5, the hurdle member 550 is substantially triangular cross-sectionally, and the top portion 551 of the hurdle member 550 has one corner 552 with a corresponding angle α, wherein the angle α is in the range of 0 to 180°.

According to another embodiment of the present invention, as shown in FIG. 12, the top portion 1251 of the hurdle member 1250 has a surface characterized by a curvature 1252. For example, the hurdle member 1250 can be an insulating liquid droplet, and the surface of the top portion of the hurdle member 1250 is at least partially spherical.

Back to FIGS. 2(a-c), the microchannel structure 200 further has a third channel 260 formed on the first surface 222 of the substrate 210 with a width, W₃, defined by a first sidewall 262 and a second, opposite sidewall 264, wherein the third channel 260 is in fluid communication with the first channel 230 at a second three-way intersection 261, wherein the third channel 260 is formed on the first surface 222 of the substrate 210 such that the hurdle member 250 is positioned between the first three-way intersection 241 and the second three-way intersection 261. The width, W₃, can be same or different from at least one of the width, W₁, and the width, W₂, wherein the width, W₁, can also be same or different from width, W₂.

The substrate 210 is formed with at least one insulating material such as a polymeric material, glass or other types of materials. In one embodiment of the present invention, the insulating polymeric material comprises PDMA.

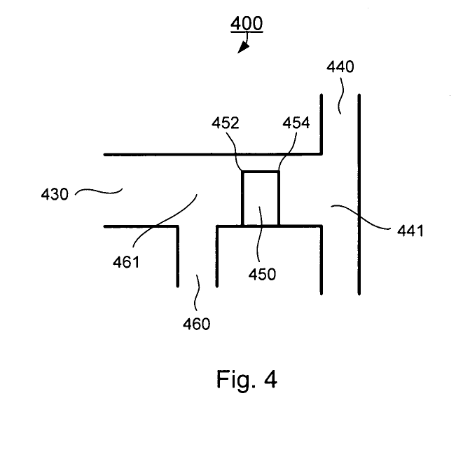

The hurdle member 250 is made from a material substantially same as the at least one insulating material of the substrate 210. Alternatively, as shown in FIG. 4, the microchannel structure 500 has a first channel 430, a second channel 440, a third channel 460, a first three-way intersection 441, a second three-way intersection 461 and a hurdle member 450, which is made from a material different from the insulating material that forms the substrate.

Referring now to FIG. 5(b), a microchannel structure 500B according to one embodiment of the present invention is shown. A first channel 530 is formed with a first portion 530a with a width, W₁, and a second portion 530b with a width, W_{1b}, which is defined by a first sidewall portion 532b and a second sidewall portion 534b, wherein the first sidewall portion 532b is located between the top portion 551 of the hurdle member 550 and the first three-way intersection 541, and the second sidewall portion 534b is located between the top portion 551 of the hurdle member 550 and the first three-way intersection 541, respectively, and wherein the width, W_{1b}, is varied at least for a portion along the first channel 530 between the top portion 551 of the hurdle member 550 and the first three-way intersection 541. As such formed, W_{1b}>W₁. More particularly, the width W_{1b} of the second portion 530b of the first channel 530 proximate to the first three-way intersection 541 is larger than the width W_{1b} of the second portion 530b of the first channel 530 proximate to the hurdle member 550.

The microchannel structure 200 further has an insulating base member 290, where the insulating base member 290 is bonded with the substrate 210 to form a sealed microchannel structure 200. The insulating base member 290 can be a glass plate.

According to the embodiment of the present invention as shown in FIG. 2(a-c), the first three-way intersection 241 is substantially T-shaped, and the second three-way intersection 261 is substantially T-shaped. They can, of course, have other shapes.

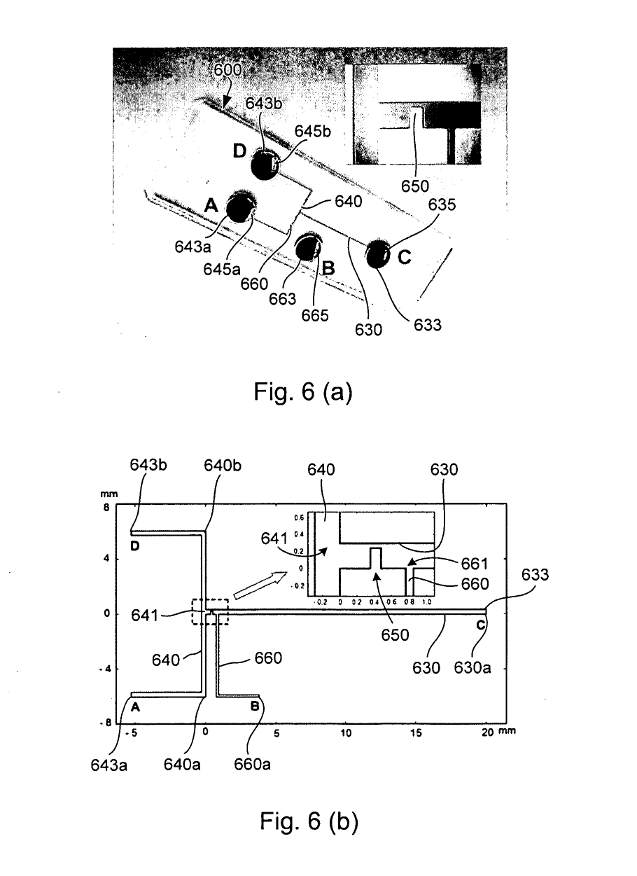

Referring now to FIGS. 6(a-b), according to another embodiment of the present invention, microchannel structure 600 has a first well 633 in fluid communication with a first channel 630 at a first end 630a of the first channel 630, a second well 643a in fluid communication with a second channel 640 at a first end 640a of the second channel 640, a third well 643b in fluid communication with the second channel 640 at a second end 640b of the second channel 640, which is apart from the first end 640a of the second channel 640, and a fourth well 663 in fluid communication with the third channel 660 at a first end 660a of the third channel 660. These wells, or ports or reservoirs, can be utilized for inputting and outputting functions. For examples, wells 633 and 663 can be used for inputting a solution, such as a buffer solution, and a particle mixture, respectively, and wells 643a and 643b can be used for collecting the separated small and large particles/cells, respectively.

Additionally, microchannel structure 600 further has a first electrode 635 configured to be positioned in the first well 633 and to be electrically connectable to a power source (not shown), a second electrode 645a configured to be positioned in the second well 643a and to be electrically connectable to a power source (not shown), a third electrode 645b configured to be positioned in the third well 643b and to be electrically connectable to a power source (not shown), and a fourth electrode 665 configured to be positioned in the fourth well 663 and to be electrically connectable to a power source (not shown), respectively. These electrodes are adapted for establishing electrical potentials or voltages in the microchannel structure.

Such one or more microchannel structures can be used to form a microfluidic chip, which in turn can be used to form a device that, for example, can separate particles or cells according to their sizes. A plurality of microchannel structures can be arranged in an array (not shown) in one embodiment of the present invention.

According to one embodiment of the present invention, microchannel structure 200 can be used as follows. A plurality of particles or cells 205 in a liquid medium is introduced into the microchannel structure 200, for example, through well 263 and/or well 233. And a direct current (DC) electrical field is applied within the microchannel structure 200 to generate a non-uniform electrical field at least around the insulating hurdle member 250 and a first voltage difference along the first channel 230 such that the plurality of particles or cells 205 is driven by the direct current (DC) electrical field along the first channel 230 and separated according to their diameters by a dielectrophoretic force corresponding to the non-uniform electrical field when the plurality of particles or cells 205 passes by the insulating hurdle member 250.

In doing so, a second voltage difference is also generated along the second channel 240 such that at the first three-way intersection 241, a first group 205b of the plurality of particles or cells 205 moves to the second channel 240 along a first direction, Y₁, and a second group 205a of the plurality of particles or cells 205 moves to the second channel 240 along a second direction, Y₂, that is different from the first direction, respectively. For the embodiment as shown in FIG. 2, Y₂ is opposite to the first direction, Y₁. Each of the first group 205b of the plurality of particles or cells 205 has a diameter that is larger than a predetermined diameter threshold, and each of the second group 205a of the plurality of particles or cells 205 has a diameter that is not larger than the predetermined diameter threshold. Thus, the plurality of particles or cells 205 is separated according to their sizes. Note that, approximately speaking, the size of a cell is proportional to (d)³, where d is the diameter of the cell. The separated particles or cells can be further collected for processing.

The present invention can be practiced to separate a plurality of particles or cells having a mix of white blood cells and red blood cells. The present invention can also be practiced to separate a plurality of particles or cells having a mix of normal cells and tumor cells. The present invention can be utilized for other applications as well.

In another aspect, the present invention relates to a method of separating particles or cells according to their sizes, wherein the size of each of the particles or cells is characterized by a corresponding diameter. In one embodiment, the method includes the steps of providing a microchannel structure having at least one channel that is defined by a first sidewall and a second, opposite sidewall and has an insulating protrusion formed on one of the first sidewall and the second, opposite sidewall, introducing a plurality of particles or cells with a conducting liquid into the at least one channel, and generating a non-uniform electrical field in the at least one channel such that when the plurality of particles or cells passes by the insulating protrusion, the plurality of particles or cells each receives a dielectrophoretic force proportional to its diameters, thereby being separable according to their sizes. The method further has the step of collecting particles or cells after the separation of particles or cells.

In yet another aspect, the present invention relates to an apparatus of separating particles or cells according to their sizes, wherein the size of each of the particles or cells is characterized by a corresponding diameter. In one embodiment, the apparatus has a microchannel structure having at least one channel that is defined by a first sidewall and a second, opposite sidewall and has an insulating protrusion formed on one of the first sidewall and the second, opposite sidewall, and means for generating a non-uniform electrical field in the at least one channel such that when the plurality of particles or cells passes by the insulating protrusion, the plurality of particles or cells each receives a dielectrophoretic force proportional to its diameters, thereby being separable according to their sizes. The apparatus may have a plurality of microchannel structures in an array.

In one embodiment, the means for generating a non-uniform electrical field includes a DC power source, a conducting liquid containable in the at least one channel and a plurality of electrodes configured to be electrically connectable to the DC power source and when connected to the DC power source, a non-uniform electrical field is generated at least in the at least one channel. The means for generating a non-uniform electrical field further has a voltage controller electrically coupled to the DC power source and the plurality of electrodes, wherein the voltage controller is capable of controlling the voltage output of each of the plurality of electrodes individually.

The apparatus further has means for receiving the plurality of particles or cells, and means for collecting the plurality of particles or cells after the separation of the plurality of particles or cells.

### EXAMPLES OF THE INVENTION

Without intent to limit the scope of the invention, additional exemplary methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as data are processed, sampled, converted, or the like according to the invention without regard for any particular theory or scheme of action.

### General Aspects of Dielectrophoresis

The present invention utilizes the principle of dielectrophoretic force. Consider a suspension of dielectric particles in a dielectric fluid. In the presence of an applied electric field, the particle and the surrounding medium are electrically polarized and the surface charge accumulates at the interfaces due to the difference in electric properties. The distribution of the surface charge of the particle gives rise to an induced dipole moment. The dipole tends to align in parallel with the local electric field. In a non-uniform electric-field, the forces acting on the opposite charges of a dipole become asymmetric. As a result, there exists a non-zero net force, called dielectrophoretic (DEP) force, acting on the particle. The induced motion of the particle due to the DEP force is known as dielectrophoresis. Using DEP, manipulation of particles can be realized by controlling the electric field without any mechanical moving part. Furthermore, different from the conventional electrophoresis that works only on the charged particles, dielectrophoresis force also acts on the electrically neutral particles, which greatly increases its biological applicability. The magnitude of the DEP force is dependent on the size and dielectric property of the particle.

Generally the particle dielectric property depends on the frequency of the applied electric field. Therefore an alternating (AC) electric field can be applied to generate DEP forces of different magnitudes and directions. The AC-DEP technique has been extensively applied in handling and characterization of micro and nanoparticle in a microsystem, such as particle trapping, manipulation, and concentration. All AC-DEP methods require complex, microfabricated, interdigitated electrode array on the chamber wall.

Most microfluidic lab-on-a-chip devices use DC electrical fields to transport liquids and cells by electroosmosis and electrophoresis. Typically DC electrokinetic microfluidic transport is realized by applying DC electrical field via electrodes inserted in the liquid reservoirs (wells) at the end of microchannels. The chips with microchannels and wells are typically made of Polydimethylsiloxane (PDMS) and glass by soft lithography method at very low cost. It should be realized that DEP does not necessarily require an AC field; it requires only a non-uniform electrical field. A non-uniform DC field in a microchannel can generate DEP. Note that the sizes of WBC and RBC are different, and the DEP force is proportional to the volume of the particle/cell. Therefore, it is possible to separate WBC from RBC by DC-DEP, or to separate the larger tumor cells by DC-DEP in a simple DC electrokinetic microfluidic chip, which is provided by the present invention.

For a non-conducting and electrically neutral particle under a low frequency AC field or a DC field, an approximate expression of the DEP force is given by

F_{DEP}=−2πεfa³(E·∀)E (1)

where εf is the liquid dielectric constant and a is the particle radius, E is the local electric field. It has been shown that this equation is valid for biological cells of spherical-shell structure. The negative sign means that the DEP force always directs to the region of the lower electric-field strength, i.e., negative DEP. The a³ in the above equation clearly indicates that the DEP force is proportional to the particle's volume.

FIGS. 1(a) and (b) illustrate a particle 105 moving under an applied DC field in a microchannel with an insulating hurdle 150. The hurdle 150 is attached on one side of the microchannel to form an abruptly narrow section 103. Since only the liquid (an aqueous solution) conducts electrical field, the narrow section 103 of the microchannel generates a spatially nonuniform DC electrical field 101 in the space near the hurdle 150.

FIG. 1b shows an enlarged view of the local electrical field 101 near the up-stream corner 152 of the hurdle 150. Under the combined effect of the electroosmotic flow (EOF) and the electrophoresis (EP), a particle 105 moves towards the entrance region of the narrow section 103 of the channel. As shown in FIG. 1b, the electric field 101 is stronger close to the corner 152 of the hurdle 150. Since the negative DEP force directs to the region of lower electric-field strength, the particle experiences a repulsive force from the corner 152 of the hurdle 150. The magnitude of the repulsive DEP force is proportional to the volume of the particle 105 and the local value of (E·∀)E, as indicated by Eq. (1). For example, the repulsive DEP force on a 15 μm particle is 27 times of that on a 5 μm particle under the same conditions. Therefore a larger particle is subject to a stronger DEP force and tends to be pushed further away from the corner 152 compared with a smaller particle. The similar DEP repulsion occurs when the particle 105 passes by the other corner 154 of the hurdle 150. As a result, the trajectory shift (in y-direction) will be different for particles of different sizes and hence particle separation by size can be expected.

### Separation of Blood Cells in a Microfluidic Device by DC-Dielectrophoresis

The DC-DEP method according to the invention may be used for separation of biological cells of different sizes, such as for separation of white blood cells from red blood cells.

### Methodology

As shown in FIG. 2, a DC-DEP blood cell separation chip 200 has a PDMS plate 220 and a glass slide, i.e., a PDMS plate 210 with the microchannel-well structure is bonded with a glass slide 290 to form a sealed microchannel structure. There are four wells 233, 263, 243a and 243b in fluid communication with the microchannel structure. DC electrical field is applied via four electrodes (not shown) inserted in these wells. A hurdle 250 is formed on one side of microchannel 230 to form an abruptly narrow section. Since only the liquid (an aqueous solution) conducts electrical field, the narrow section of the microchannel 230 generates a spatially non-uniform DC electrical field in the liquid near the hurdle 250. The mixed larger and smaller cells 205 in a liquid medium is introduced into channel 230 (inputting channel) from channel 260 (i.e., inputting branch). As explained above, the negative DC-DEP force at the corners 252, 254 of the hurdle 250 pushes larger cells 205b more than smaller cells 205a, and hence generates different trajectories for larger and smaller cells once they pass the hurdle 250. After the hurdle 250, a T-shaped channel structure 241 may be used so that the separated larger cells 205b and the separated smaller cells 205a are drawn into separate cell collection wells by electrokinetic flows.

Since the particles' trajectories after the hurdle 250 and hence the final separation efficiency are also coupled with the electrokinetic flows of the liquids in different channel branches, voltage output may be adjusted to obtain the optimal voltages applied at the four electrodes.

If desired, this design may be integrated to existing lab-on-a-chip devices that require separated white blood cells (WBC), and extend this method into a high throughput technology to handle a large volume of blood sample. For instance, using micro-fabrication method one can build hundreds of parallel microchannels on a single chip, so that separation of blood cells from a larger volume of blood sample can be achieved.

### Modeling and Numerical Simulation

To find the optimal design parameters (e.g., the hurdle size and position) and the optimal controlling parameters (e.g., applied voltages) for such a cell separation chip, it is desirable to understand the electrokinetic motion of cells in the microchannel as illustrated in FIG. 2, and the influences of different parameters on the cells' trajectory. Generally, the cell transport in a microchannel involves both the electroosmotic flow (liquid motion) and the electrophoresis (particle motion relative to the liquid). Furthermore, when cells pass the hurdle, the non-uniform electrical field and hence the DC-DEP force has to be considered. So far, most existing works of electrokinetic motion of particles are limited for simple geometries such as electrophoresis of a particle in a quiescent unbounded liquid, or near a planar wall. The cell motion in a 3D microchannel such as the one in FIG. 2 is transient and three-dimensional. Predicting the cells' trajectories require a complicated theoretical model and numerical simulation, the information of which can be found in several of the references.

A theoretical model for the cell transport processes in the microchannel under an applied DC electrical field includes: (1) the Laplace equation for the applied electrical field; (2) 3D equations of motion to describe the flow field of the liquid; and (3) Newton's 2^{nd} law equation, including the electrophoresis force, the dielectrophoresis force, the flow friction force, to determine the motion (velocity and trajectory) of the particle. As the system used here involves buffer solutions with high ionic strength, the thickness of the electric double layer fields around the cells and channel walls are less than 10 nm. Therefore, the thin double layer treatment may be used, i.e., the double layer causing electroosmotic flow may be considered as a slip velocity boundary condition for the equation of motion. The complexity and the coupling effects involved in these equations and the boundary conditions require developing an efficient numerical method.

In the numerical simulation, one of challenges is to avoid re-meshing. an ALE (arbitrary Lagrangian-Eulerian) approach for particle tracking, based on a generalized Galerkin finite element formulation, may be used to solve over a 3D unstructured tetrahedral mesh. As a particle moves, the mesh becomes distorted and new meshes are required at specific time steps in order to capture this motion. These re-meshing steps are very time-consuming, rendering this method unsuitable for tracking the particle over large distances. Employing the Chimera or overlapping grid scheme may solve the electrophoretic motion of a particle in a microchannel. The advantages of this method are that it eliminates the need for computationally expensive re-meshing steps, and it simplifies the procedures for solving the discretized equations by using structured rather than unstructured grids.

### Methods and Materials

One basic microchannel structure of the cell separation chip is shown in FIG. 2. There are four reservoirs (wells) connected at the ends of the four microchannel branches. The PDMS (polydimethylsiloxane) microchannel can be fabricated following the soft lithography protocol.

Fluorescent (carboxylate-modified) polystyrene particles of different sizes, 6 μm, 10 μm and 15 μm in diameter (Bangs Laboratory Inc.) may be used as sample particles for fundamental studies. These particle sizes are similar to the size of typical biological cells such as the red blood cells and the white blood cells.

Before use, the channel and all the wells may be primed with the 1 mM sodium carbonate buffer solutions. Then the cells or particle mixture may be introduced into the well with a syringe. A high-voltage DC power supply (Labsmith HVS448) may be used to drive the liquid flow though the microchannel structure by platinum electrodes submerged in each well. This power supply can provide and control the voltage outputs of each of the four electrodes independently. Following the results of the numerical simulations as guidance, the voltage applied to electrodes may be carefully adjusted to realize that the liquids and the cells/particles in the inputting branches always move towards the hurdle and eventually flow into the two separation branches. Because the whole separation process can be completed within 30 seconds, and the electroosmotic flow rate in the microchannel is small, the effect of the pressure-driven flow can be minimized by using sufficiently large well size and by carefully balancing the liquid level in four wells before each experimental run. The cell/particle motion may be monitored by an inverted optical microscope (TE2000-U, Nikon Inc.) and recorded by a progressive CCD camera (QImaging, British Columbia, Canada).

The parameters that have effects on the cell/particle separation include: (1) design parameters, such as the channel's dimensions, the hurdle's size and position; and (2) operation parameters, i.e., the applied voltages at different electrodes. The experimentally measured cells' trajectories with different design parameters and operation parameters may be compared with the predictions of numerical simulations. Several sets of optimal design parameters and operation parameters may be determined. The microfluidic chip of the invention may include a plate with hundreds of parallel microchannels, making it feasible to perform high-throughput of on-chip blood cell separation. Conventionally, most lab-on-chip applications are interested only in the treatment of very small amount of samples. The typical speed of the cell motion in the microchannels is about 1000 μm/s. Assuming the average cell size is 10 μm and considering the fact of multiple cells moving parallel through the channel, the invention may make it feasible to treat 200˜300 cells/second or 12000˜18000 cells/min in one microchannel. For instance, one can use the design provided in the present invention to build a chip with 10 parallel channels for performing high-throughout of on-chip blood cell separation.

### Example 1

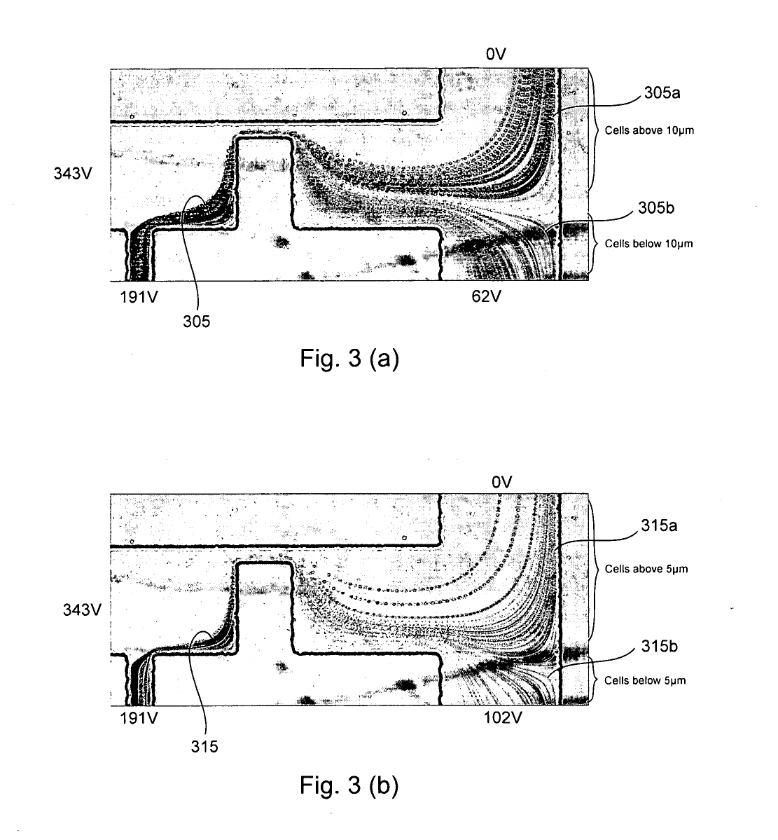

The present invention has been used to separate different white blood cells. In the experiments, a 50 μl volume of blood was mixed with 50 μl of a Red Blood Cell Lysis Buffer (Caltag, Burlingame, Calif.) to lyse the red blood cells, and then diluted with 500 μl of de-ionized water (this protocol fixes WBC in the sample, and lyses RBC). 10 μl of this sample solution was loaded to the sample well on the chip by a micro-pipette. 10 μl of this sample solution contains approximately 8,000 cells (granulocytes, monocytes, and lymphocytes) and approximately 100,000 small components (platelets, RBC debris, etc). By adjusting the applied voltages at different electrodes inserted in the wells at the ends of the microchannels, the inventor separated the white blood cells at a specified cell size, which is corresponding to a predetermined diameter for the cell such as 10 μm as shown in FIG. 3 as an example.

The applied electrical field has negligible effects on the cells, other than generating the cell motion. This can be appreciated by comparing the applied electrical field strength with the electrical field strength of the cells' electrical double layer (EDL) field (i.e., the field around each cell generated by the natural surface electrostatic charge). The typical EDL field strength is 100 mV/10 nm=100,000V/cm, while the applied electrical field ranges from 10 V/cm to 100 V/cm.

### A Microfluidic Method for Isolation of Circulating Tumor Cells

### Methodology

The methodology used for isolation of circulating tumor cells is similar to that illustrated in FIG. 2. Initially, a blood sample solution 205 containing a few larger tumor cells 205b and many smaller normal blood cells 205a may be introduced into a sample well. Under an applied electrical field, the mixed larger and smaller cells will be introduced with the flow into the main channel 230 from a channel 260 (inputting branch) through a three-way intersection 261. As explained above, the negative DC-DEP force at the corners 252, 254 of the hurdle 250 will push the larger tumor cell 205b more than the smaller normal cells 205a, and hence generate different trajectories for the larger tumor cell and the smaller normal cells once they pass the hurdle 250. After the hurdle, a T-shaped three-way intersection 241 will allow the separated larger tumor cells 240 and the separated smaller normal cells 240 to be draw into separate cell collection wells by electrokinetic flows.

### Modeling and Numerical Simulation

To separate the larger tumor cells from the smaller normal cells, it is necessary to be able to control the cells' trajectories after the hurdle. The cells' trajectories depend on the DEP force acting on the cells and hence on the hurdle' size and position, the channel's size, and the applied electrical field.

Using the theoretical model and the numerical method stated above, the method(s) of the invention can be used to conduct extensive numerical simulations of the on-chip processes of separating the larger tumor cells from the rest, under various conditions. The results and findings of these numerical experimental studies can allow us to determine the optimal design parameters (e.g., the hurdle size and position) and the optimal controlling parameters (e.g., applied voltages) for such a tumor cell separation chip.

### Methods and Materials

The basic design of a circulating tumor cell separation chip is similar to the one shown in FIG. 2. There are four reservoirs (wells) connected at the ends of the four microchannel branches. The PDMS (poly-dimethylsiloxane) microchannel may be fabricated following the soft lithography protocol.

Before the experiment, the channel and all the wells may be primed with the 1 mM sodium carbonate buffer solutions. A mixture of normal blood cells and tumor cells, after being processed appropriately, may be introduced into the sample well with a digital micro-pipette and a Nikon cell injector. A high-voltage DC power supply (Labsmith HVS448) may be used to drive the liquid flow though the microchannel network by platinum electrodes submerged in each well. This power supply can provide and control the voltage outputs of the four electrodes independently. Following the results of the numerical simulations as guidance, the voltage applied to the electrodes may be carefully adjusted to realize that the liquids and the cells in the inputting branches 260 will move towards the hurdle 250 and eventually flow into the two separation branches 243a, 243b. Because the whole separation process can be completed within 60 seconds, and the electroosmotic flow rate in the microchannel is small, the effect of the pressure-driven flow can be minimized by using sufficiently large well size and by carefully balancing the liquid level in four wells before each experimental run. The cell motion may be monitored by an inverted microscope (TE2000-U, Nikon Inc.) and recorded by a progressive CCD camera (QImaging, British Columbia, Canada) and a digital imaging system.

The parameters that may have effects on the tumor cell separation include: (1) design parameters, such as the channel's dimensions, the hurdle's size and position; and (2) operation parameters, i.e., the applied voltages at different electrodes. The experimentally measured cells' trajectories with different design parameters and operation parameters may be compared with the predictions of numerical simulations. Several sets of optimal design parameters and operation parameters may be determined.

To develop an electrokinetic based microfluidic chip to separate circulating tumor cells from blood with high sensitivity, one can analyze the cells in each collection well using the microscope to examine the number of the isolated tumor cells. The preferred separation efficiency is to reach about 100% isolation/separation of the circulating tumor cells.

The microfluidic chip of the present invention is the first device developed that have applicability in the DC-DEP separation of circulating tumor cells, and may prove its ability of separating one tumor cell out of 10,000 or more normal cells. The method disclosed in the invention is not limited to separate tumor cells in blood; it can be applied to separate tumor cells in other bio-fluids as well, such as sputum and urine. It may be used to separate various types of tumor cells. In addition, the device and the method of the invention may be used for high throughput, for example, by using multiple parallel microchannels in an array on a single chip. Further, the invention utilized be made to be a fully-automatic, practical and effective tool for biomedical research.

In summary, in one aspect, the invention provides a new electrokinetic based microfluidic method to separate the larger tumor cells in blood samples. The separation is performed on a chip with a size of a microscope glass slide, is a single step process and operated in a continue flow mode. The device according to the invention has no mechanical moving parts and no filters. No any labeling is required. This method can also be applied to separate circulating tumor cells in other forms of body fluids.

In one embodiment, the DC-DEP method of the invention may further include a step of providing a diverging microchannel section immediately after the hurdle, the diverging microchannel section being located on the opposite side of the hurdle and connecting the narrow section to the outputting channel.

Referring to FIG. 4, the DC-DEP particle/cell separation chip 400 uses a rectangular hurdle 450. The design in FIG. 5 uses a triangle shaped hurdle 550, instead of a rectangular hurdle. In the case of a rectangular hurdle 450 of FIG. 4, the DC-DEP force at the first corner (upstream) 452 of the rectangular hurdle 450 will push larger cells more and push smaller cells less, and hence create the initial separation. As a result, the larger cells will move away from the second corner (downstream) 454 of the hurdle 450, in comparison with the smaller cells. The larger cells will receive much less interaction from the second corner 454. However, the smaller cells will be pushed by the DC-DEP force at the second (downstream) corner 454 of the rectangular hurdle 450, and they will be pushed closer to the larger cells. Therefore, the overall effect of the DC-DEP separation may be reduced. Using a triangle hurdle may help reduce or get rid of the second corner effects.

The design in FIG. 5B uses a diverging microchannel section immediately after the hurdle 550, which will create diverging stream lines, and help the separated lager cells move further away from the smaller cells.

### Separation of Microparticles by Size with Direct Current-Dielectrophoresis

### Materials and Methods

Referring now to FIG. 6, there are four branches connected to four different reservoirs. Wells or reservoirs B (663) and C (633) are for inputting the particle mixture and the buffer solution, respectively. Wells or reservoirs A (643a) and D (643b) are for collecting the separated small and large particles, respectively. Branches A (640), C (630), and D (640) are 300 μm in width. Branch B (660) is 90 μm in width. All of the branch channels are 45 μm in depth (in z-direction). The kernel structure is a rectangular block 650 (240 μm×130 μm) located between the first three-way intersection 641 and the second three-way-intersection 661. The polydimethylsiloxane (PDMS) microchannel was fabricated following the soft lithography protocol.

Fluorescent (carboxylate-modified) polystyrene particles of three different sizes, 5.7, 10.35, and 15.7 μm in diameter (Bangs Laboratory), were used as sample particles. These particle sizes are similar to the size of typical biological cells such as the red blood cells and the white blood cells. The particles were supplied in the form of 1% suspension in pure water. These particle solutions were further diluted with the 1 mM sodium carbonate buffer (Na₂CO₃/NaHCO₃) solutions. The number density of particle was normally about 10⁵/mL. Since the mass density of the particles was slightly greater than that of water (nominal density is 1.05 g/mL), the particle solutions were gently vibrated prior to use to prevent sedimentation.

Before the experiment, the channel and all the wells or reservoirs were primed with the 1 mM sodium carbonate buffer solutions. Then the particle mixture was introduced into reservoir B (663) with a 1-mL plastic syringe. A high-voltage DC power supply (Glassman High Voltage, High Bridge, N.J.) was used to drive the fluid flow through the microchannel structure by platinum electrodes submerged in each reservoir. A custom-made voltage controller was used to adjust independently the voltage output of each of the four electrodes. In the experiments, electrode D (645b) was always grounded. The voltage outputs to electrodes A (645a), B (665), and C (635) were carefully adjusted to realize that the fluids in the inputting branches B (660) and C (630) always moved to the block 650 and flowed into the separation branches A (640a) and D (640b). The pressure-driven flow was minimized by carefully balancing the liquid level in four reservoirs before each experimental run.

The particle motion was monitored by an inverted optical microscope (Nikon Canada) and recorded by a progressive CCD camera (QImaging, Burnaby, British Columbia, Canada). The camera was operated in video mode at a frame rate of 11.4 frames per second. The acquired images (viewed from the top) had a resolution of 640×484 pixels. The reading error to determine the particle positions is about ±2 pixels which corresponds to actual dimension of ±5.4 mm.

### Results

In the numerical simulation, the zeta potential of the PDMS channel wall was set to 220 mV. The electrophoretic mobilities of the 5.7 and 15.7 μm particles were fixed as 3.3×10⁻⁸ and 3.7×10⁻⁹ m²s⁻¹V⁻¹, respectively, which were based on an independent measurement in a straight channel using the same buffer solution. Because the ionic concentration of the working solution is very low, the liquid properties are not different from that of deionized (DI) water, that is, dynamic viscosity 1.0×10⁻³ kg\ddot{}m⁻¹s⁻¹, density 998 kg/m³, and electrical permittivity 6.96×10⁻¹⁰ C·V⁻¹m⁻¹.

### A Typical Case

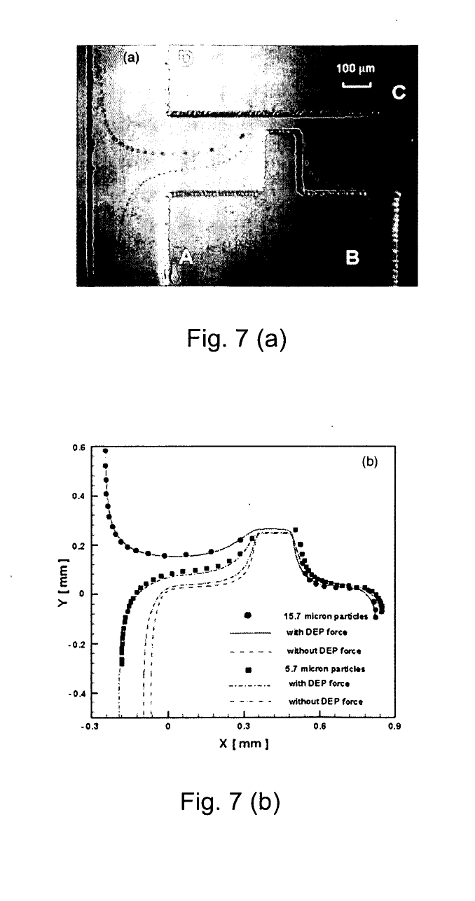

The magnitude of the particle trajectory deviation is proportional to the DEP force acting on the particle, and hence the particle volume. Therefore, the trajectories of the particles of different sizes can be diverted into different streams after they pass the block 650. A typical case of separation of 5.7 μm particles and 15.7 μm particles is shown in FIG. 7(a), which was obtained by superposing a series of consecutive images of the moving particles. Initially the particle mixture came out as a single stream from the inputting branch B 660. Then the main stream of the buffer solution from branch C 630 squeezed the mixture and forces the particles to move closely to the block corner. After the particles passed through the gap between the block 650 and the channel wall, their trajectories were changed. The trajectory deviation for a larger particle was greater than that for a smaller particle because of the different magnitude of the DEP force they experience at the block corners. Thus, the single mixture stream was separated into two. By adjusting the voltage at electrode A, the larger particle moved into the separation branch D 696, while the smaller particle moves into the other separation branch A 698. Eventually the particle mixture is continuously separated into two different reservoirs. The particles in reservoirs D 643b and A 643a were pure 15.7 and 5.7 mm particles, respectively.

The individual trajectory of the particles can be predicted by using the numerical model previously developed by us. FIG. 7b demonstrates the comparison between the simulation results and the experimental results for the separation of 5.7 and 15.7 μm particles. The dotted symbols are digitized positions of the particles based on FIG. 7a. The smooth curves are the simulated trajectories. According to Equation: F_{DEP}=−πcε_{f}a³∀|E|², we introduced previously a correction factor c to account for the finite-size effect to the actual DEP force. It was found that simulation results could give a close match to most of the experimental results by setting c=0.3 for 5.7 μm particle and c=0.4 for 15.7 μm particle. One may infer that the size-related correction factor c should approach unity if the particle size goes to infinitely small. It seemed that larger particle should have a smaller correction factor (further from unity). This might be true in an unbounded domain. However, when the distance from a particle to channel wall was very close, such as in the order of particle size, the electric field would be locally perturbed significantly due to particle-wall electrical interaction. It could be even more significant when a particle was located near the block corner where the DEP force was greatest. Thus, the smaller particle might be more affected by the particle-wall electrical interaction and could lead to a greater deviation from unity. It was challenging to find out, considering the effect of particle-wall interaction, a complete functional dependence of the correction factor.

In order to show that the trajectory deviation was indeed because of the DEP force, we also removed the term F_{DEP} from Eq. Fₐₚₚ=F_{EP}+F_{DEP}, and computed the resulting particle trajectories, as shown by the other two curves in FIG. 7B. As anticipated, the particle trajectories before and after the block are symmetrical and apparently there was no deviation at downstream, if there was no DEP effect.

### Effect of Voltage Output

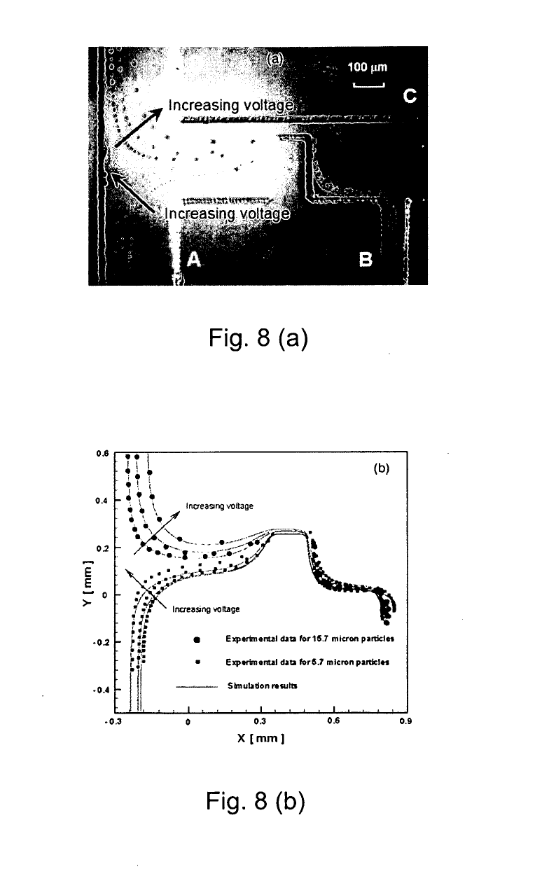

The voltage outputs of the four electrodes for different situations are specified in Table 1. Since the voltage output of the four electrodes satisfied V_{C}>V_{B}>V_{A}>V_{D}, we defined the highest output V_{C} as the system voltage level. FIG. 8 shows the separation of 5.7 and 15.7 μm particles at different voltage levels from about 500 to 900 V. FIG. 8A shows the superposed particle trajectories and FIG. 8B shows the comparison of the experimental and simulation results. It was demonstrated that the magnitude of trajectory deviation for both larger and smaller particles increased with increasing the voltage level.

This was because the DEP force is proportional to the gradient of the electric-field intensity, ∀|E|². The other direct effect under the higher voltage level is that the particle velocity (and hence the separation process) becomes much faster. As shown in FIG. 8, at the same frame rate, the distance between consecutive particle positions became greater under higher voltage, implying that the particle moving speed became faster. This was because the EOF was enhanced under a strong electric field. However, we could not infinitely increase the voltage to speed up the separation process because in the practical application the biological samples are subjected to lyses under too strong electric field. The other possible side effect associated with too high voltage is the Joule heating, which may burn the channel and the biological samples as well.

As shown in FIG. 8B, a large discrepancy between experimental result and simulation was observed for smaller particles well after the block 650. It has been proved by the simulation that the predicted particle trajectory after the block is sensitive to the value of the correction factors. Except for the accuracy of the correction factor as discussed in one of the references, there are many other likely sources of error, such as uncertainty in corner radii, sidewall slope, surface roughness, uncertainty of particle positions in channel B 660, pressure-driven flow due to head differences, limited computational domain, boundary conditions, etc. All these physical or geometrical uncertainties might contribute to the discrepancy between experiment and simulation.

Other than the voltage level V_{C}, the voltage output at electrodes A and B is also important to realize the separation. The major function of the electrode B in the inputting reservoir was for driving the particle mixture into the block region, so that V_{B} should not be very small. Otherwise the EOF will be directed to flow back into branch B 660 and the particle mixture cannot be successfully introduced into the channel network. In this experiment, we found it wise to keep V_{B} around 50% of V_{C}, as shown in Table 1.

TABLE 1

| | |
|---|---|
| Particle | Voltage output |
| size, μm | at electrodes, V |

| I | II | A | B | C |
|---|---|---|---|---|
| 5.7 | 10.35 | 203 | 280 | 501 |
| | | 305 | 393 | 707 |
| | | 329 | 503 | 904 |
| 5.7 | 15.7 | 54 | 244 | 502 |
| | | 76 | 344 | 703 |
| | | 99 | 445 | 909 |
| 10.35 | 15.7 | 95 | 280 | 505 |
| | | 95 | 388 | 699 |
| | | 124 | 505 | 909 |

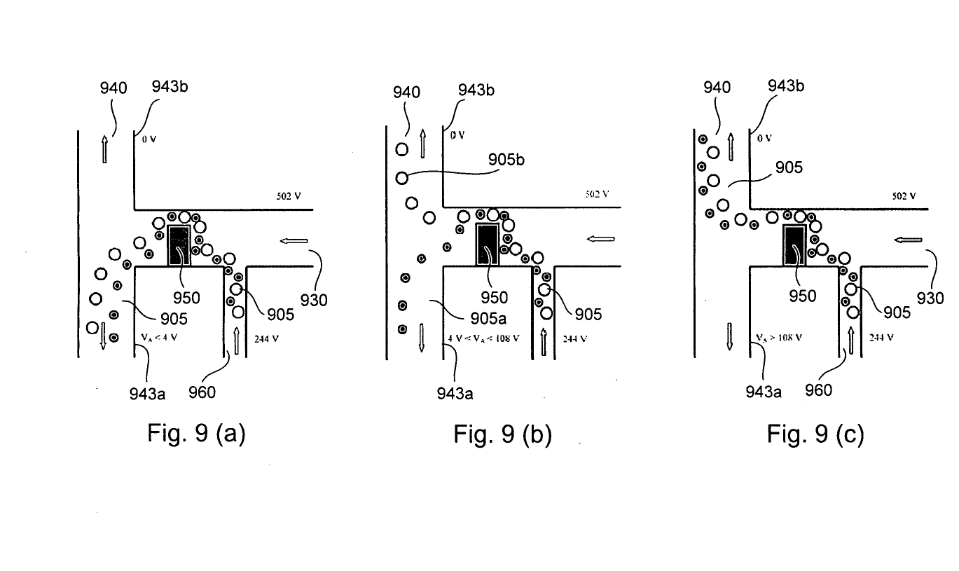

The applied voltage at electrode A was for controlling the flow streams and hence the particle motion after the block. According to our numerical simulation, the electric field and the flow field had a similarity and showed the same spatial profile. The particles experienced strong DEP force near the block corners where the electric field was highly nonuniform. However once the particles moved out of the block region, the electric field became uniform and there was no DEP force acting on the particles any more; only the Stokes frictional force and electrophoretic force are present. Therefore, the particles always moved following the streamlines. It has been shown that the single stream of particle mixture is separated into two different streams after the block 650. In this experiment, it was found that there existed an effective range of V_{A} in order to realize the separation, which was bounded by two threshold values. As shown by the schematic illustration in FIG. 9, when V_{A} was lower than a threshold voltage 4 V, all of the particles 905 moved into branch 943a (FIG. 9A); whereas when V_{A} was higher than the other threshold voltage 108 V, all of the particles moved into branch 943b (FIG. 9C). The effective separation only occurred when V_{A} fell between these two threshold voltages (FIG. 9B), where smaller cells 905a move to branch 943a after passing the block or hurdle member 950 from main channel 930 and larger cells 905b move to branch 943b. The values of the above two threshold voltages were based on the numerical simulation for 5.7 and 15.7 μm particles at the voltage level of 500 V. The real values may be slightly different in the experiments. It can be reasonably inferred that the threshold voltages are dependent on the channel configuration and the sizes of the particles. They can be determined by experimental calibration.

### Sensitivity of the Separation

The above observations were all based on the separation of 5.7 and 15.7 μm particles. To test the sensitivity of this separation method, we also conducted experiments using particle mixtures of other size combinations, such as 5.7 with 10.35 μm, and 10.35 with 15.7 μm. By adjusting the voltage output of the electrodes, we successfully realized the separation of above two particle mixtures using the same channel configuration. The voltage outputs were specified in Table 1. This means that the DC-DEP method according to the invention can separate the particles with different size differences. As one of its major advantages, separating target particles of a different size can be realized simply by adjusting the applied voltages. Channel reconfiguration, such as a new design or modified dimensions, is not required.

### Visualization of the Fluid Flow

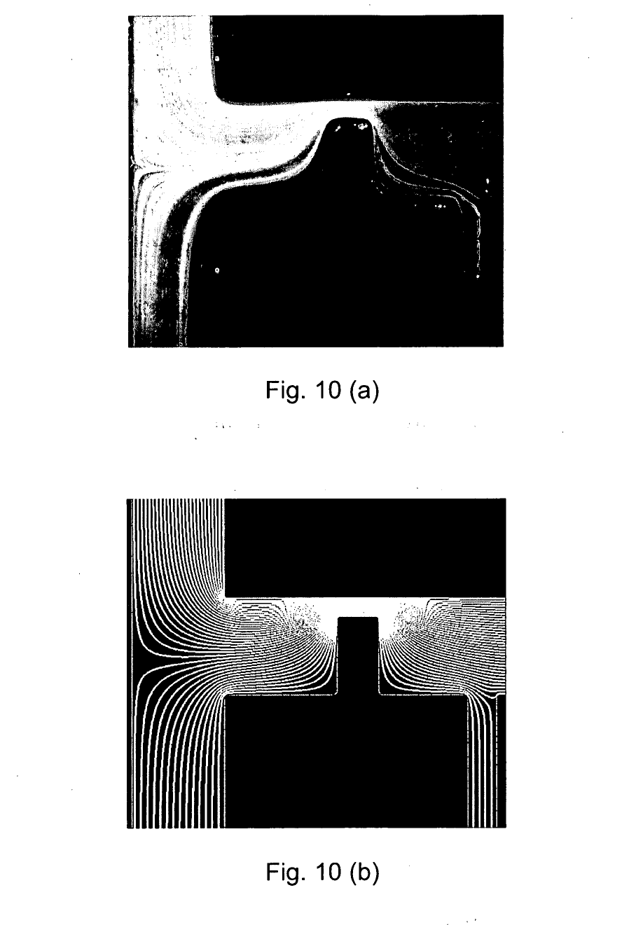

FIG. 10a shows the streamlines of the EOF near the block 650 which was obtained by using 1 μm fluorescent polystyrene particles suspended in 1 mM sodium carbonate buffer solution. This image was taken by a CCD camera at an exposure time of 4 s. It could be seen that fluid from the particle input branch B 660 was squeezed to move closely near the wall of the block 650 by the main flow from the buffer input branch C 630. This is the purpose of branch C 630. This design can ensure that the particles move sufficiently near the block corners where they will experience strong DEP force. Thus, the particles will have the greatest trajectory changes after they pass the block region 650.

The electrostatic field and the EOF field in the microchannel were simulated using FEMLAB® (Comsol). The simulated streamlines are shown in FIG. 10b. The simulation results fit reasonably well with the experimental results and were used for the subsequent simulation of the particle trajectory.

### Example 2

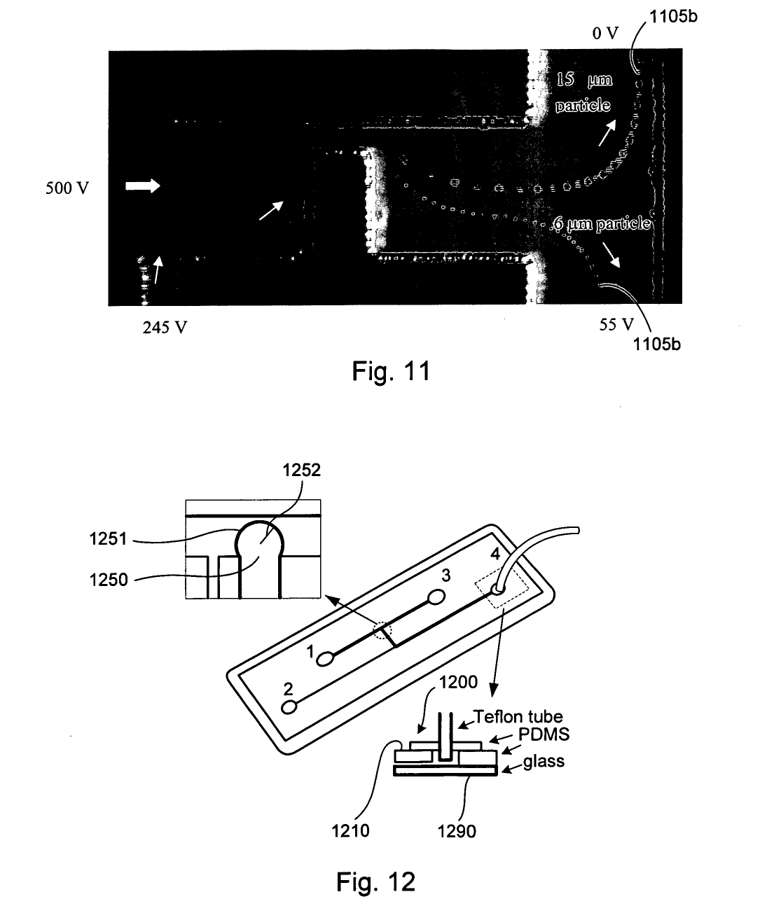

FIG. 11 shows trajectories of 6 μm and 15 μm polystyrene particles by superimposing a series of sequential microscopy images obtained from an experiment. The microchannel (made of PDMS and glass by soft lithography) in this case was 300 μm in width and 40 μm in depth (perpendicular to the paper). The narrow section of the microchannel was 60 μm in width. We found that when the mixed particles approached the narrow gap from the side of the hurdle, the DC-DEP effect produced the best separation. As set forth above, when larger particles and smaller particles move closely over the corner of the hurdle where the non-uniform electrical field gradient is the strongest, the larger particles are subject to a stronger DEP force and are pushed further away from the corner compared with smaller particles. Consequently the larger particles and smaller particles follow separate trajectories after passing the hurdle. Applying appropriate voltages to induce the downstream flows to the two separating channel branches, the large particles and the small particles could be separated and collected in separated wells. Using the method of the invention, we could separate particles with a size difference as small as two microns.

### DC-Dielectrophoretic Separation of Particles Using an Oil Droplet Obstacle

Experiments were conducted in a microfluidic chip illustrated in FIG. 12, which is composed of a PDMS upper substrate 1210 and a glass lower substrate 1290. This microchannel structure 1200 has four 40 μm deep branch channels (correspondingly, four reservoirs filled with particle solution, distilled water or oil): 515 μm wide and 12.5 mm long channel 1 reservoir supplies the system with the distilled water; 131 μm wide and 25 mm long channel 2 reservoir transports particles suspended in distilled water to the droplet region (see the inset of the zoom-in view in FIG. 12); 515 μm wide and 12.5 mm long channel 3 reservoir collects the separated particles after passing around the droplet; and 518 μm wide and 25 mm long channel 4 reservoir transports oil. The oil is supplied through the Teflon tubing (762 μm id/1.22 mm od) connected to a plastic syringe (see the inset of schematic in FIG. 12).

### Microfabrication

The microchip was fabricated in PDMS using a standard single-layer soft lithography technique. A detailed procedure is described in one of the reference, except for the oil connection shown in FIG. 12, which required few additional fabrication steps. As oil was forced through the microchannel by applying pressure with a syringe pump, the oil reservoir had to be sealed, unlike the other three reservoirs containing aqueous solutions. A square piece of a thin PDMS layer with a punched through opening for the Teflon tube was used as a lid for the oil reservoir. Both the PDMS chip and the lid were treated in oxygen plasma (plasma cleaner PDC-32G, Harrick Scientific, Ossining, N.Y.) for 30 s and then bonded to each other. Prior to that, the top surface was covered, except for the region where the lid was to be bonded. In this way, regions around the other three reservoirs kept the hydrophobic property, which prevented liquid in the reservoirs from spilling out. After the chip fabrication, channels were filled with distilled water and the next step was forming of an oil droplet. Teflon tubing connected to a 1 ml plastic syringe containing mineral oil (Nujol, Applied Biosystems) was initially filled with oil and then press fitted into the lid opening. Using a custom made syringe pump, mineral oil was pushed into reservoir 4 and then slowly forced through branch channel 4, until it reached the T-junction where it formed the droplet 1250, as shown in the zoom-in view in FIG. 12. The droplet diameter was approximately 600 μm.

### Experimental Procedure

Fluorescent (carboxylate-modified) polystyrene particles of three different nominal sizes, 1 μm, 5.7 μm and 15.7 μm in diameter (Bangs Laboratory Inc.), were used as model particles for dielectrophoresis. Particles originally suspended in water at a 1% weight ratio were diluted with DI water 50 times. The suspension was gently vibrated prior to use to homogenize the particle distribution and then introduced into reservoir 2. The EOF was generated by using a high-voltage DC power supply (Glassman High Voltage Inc., High Bridge, N.J.) and platinum electrodes submerged in each water reservoir. Reservoir 3 was always grounded, while the voltage outputs of the other two reservoirs were adjusted by a custom-made voltage controller, so that voltage 1 was always higher than voltage 2. In this way, the particles coming from branch 2 were suppressed with the EOF from branch 1. They were forced to move close to the channel wall and to approach the droplet at its base. This kind of particle focusing was necessary, as all particles needed to approach the droplet at the same level, so that the difference in their trajectory shift after the droplet was more obvious. Pressure-driven flow was eliminated before each measurement by balancing the liquid level in reservoirs.

Particle motion was visualized using a Leica DMLM fluorescence microscope with 10× and 16× objectives, the appropriate filter set and a 100 W broadband mercury lamp. Images were recorded with a 12 bit Retiga-1300 cooled digital CCD camera (Pulnix America Inc., Sunnyvale, Calif.) and OpenLab 3.1.5 image acquisition software. All images were recorded with 80 ms exposure time. The acquired images have the resolution of 640×512 pixels, with each pixel representing 2 μm and 3.3 μm square in object plane for 16× and 10× objective, respectively.

### Results

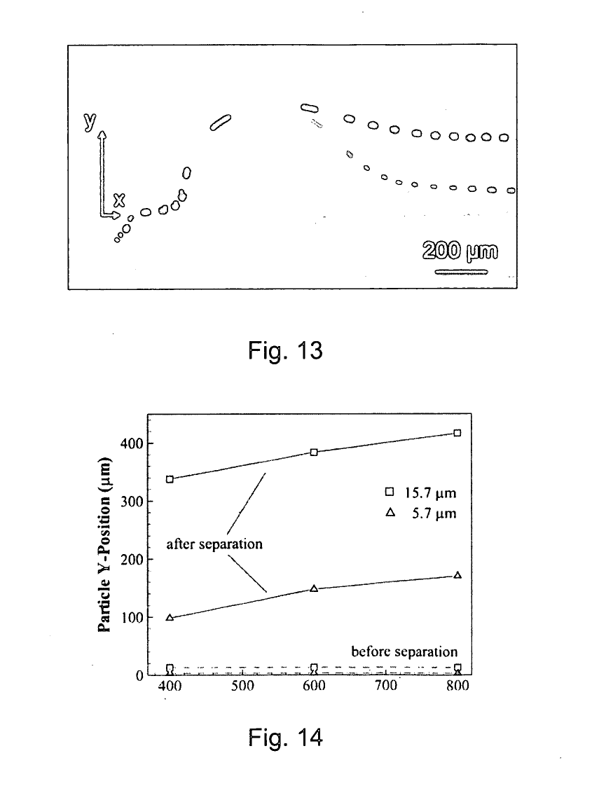

As described by equation (1) F_{DEP}=−2πεfa3(E·∀)E, particles of different sizes experience different magnitudes of the dielectrophoretic force. FIG. 13 shows the alteration of the trajectories of 5.7 μm and 15.7 μm particles for the gap of 45 μm between the droplet and the channel wall. This figure was obtained by superposing a series of consecutive images of the moving particles while 400 V was applied to reservoir 1 and 160 V to reservoir 2. Both 5.7 μm and 15.7 μm particles had almost an identical trajectory at the upstream side of the droplet, which was achieved by focusing the particles from branch 2 close to the droplet base, as explained previously. Particle trajectories were separated after passing the droplet: the 15.7 μm particles moved from y=13 μm just before the droplet to y=338 μm downstream, while the 5.7 μm particles shifted from y=3 μm to y=98 μm. Since the magnitude of the DEP force is proportional to the particle volume, the 15.7 μm particle experienced approximately a 20 times stronger DEP force than the 5.7 μm particle did. In the mean time, the Stokes resistance exerted on the 15.7 μm particle was 2.8 times of the 5.7 μm particle so that the trajectory shift of the former particle was essentially 7.5 times of the latter if both particles were shifted by dielectrophoresis within the same time period. In the experiment, however, 15.7 μm particles moved almost 2 times faster than 5.7 μm particles, resulting in a longer residing time for smaller particles. Incorporating all these effects, the actual trajectory shift of 15.7 μm particles was estimated as 3.75 times that of 5.7 μm particles. This estimation is in good agreement with the ratio of the observed particle trajectory shift, i.e., (338−13)/(98−3)=3.42. The demonstrated difference in the trajectories can be utilized to separate the particles, by allowing them to flow into two different reservoirs after the trajectory shift (in the y direction). Easier separation is attainable if the distance between the trajectories is larger. The effectiveness of the separation depends on experimental parameters such as electrical field, droplet size and particle size. These parameters will be examined in the following sections.

### Electric Field Effect

The electric field strength in the gap region was varied by tuning the electric potentials applied at reservoirs 1 and 2. However, the ratio of these two voltages was kept constant as the best particle focusing was observed when voltage 1 was around 2.5 times higher than voltage 2. FIG. 14 compares the y-coordinates of 5.7 μm and 15.7 μm particles when their trajectories became steady downstream i.e. parallel to the channel sidewalls. The y-coordinates of both particles before the droplet are also included for reference. It is apparent that all particles approached the droplet at its base, at approximately zero y-coordinate (3 μm for the 5.7 μm particles and 13 μm for the 15.7 μm particles). They were repulsed from the gap at the top of the droplet while flowing through the nonuniform field. The stronger the electric field, the greater the trajectory shift achieved. However, it should be noted that the voltage increase enhanced the trajectory shift of both types of particles, but did not really enhance their separation after the droplet. For all applied voltages, the difference between the downstream y-coordinate of the 5.7 μm and 15.7 μm particles remained around 240 μm.

### Droplet Size Effect

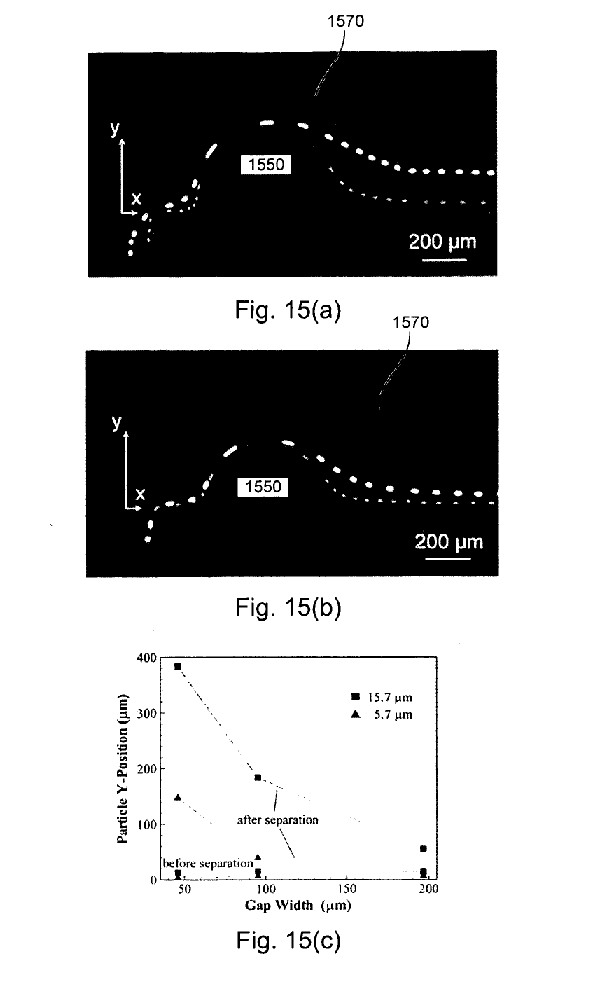

In this novel DC-DEP separation design the size of the insulating obstacle can be varied, so the electric field gradient was easily controlled by changing the droplet size. When the droplet size was increased, the electric field was more compressed between the droplet and the channel wall, inducing a higher DEP force. Particle separation for three different gap sizes, 46 μm, 95 μm, and 197 μm, was tested. The applied voltages at reservoirs 1 and 2 were fixed at 600 V and 235 V, respectively. Particle trajectories FIG. 15A (gap width of 95 μm) and 15B (gap width of 197 μm) show that all particles were focused to the base of the droplet, y=6 μm for the 5.7 μm particles, and y=15 μm for the 15.7 μm particles. FIG. 15C shows that as the gap size decreased four-fold from 197 μm to 46 μm, the distance between the trajectories of the small and big particles increased 5.5-fold from 43 μm to 236 μm. With this large distance, as large as half of the channel width, the big and small particles could be easily separated downstream into two branches/reservoirs.

Unlike the aforementioned electric field effect that took place in the whole chip, the change in the droplet size influenced only the electric potential distribution around the droplet. Consequently, large electric field gradients could be generated locally by applying small voltages. In addition, as the particle diameter was comparable to the gap width, the electric potential field was further distorted by the particles passing through the gap. These two consequences should explain why the separation between the small and large particles increased when the gap width was decreased. At a small gap width, however, we noticed an appreciable change of trajectory shift when multiple particles of the same or different size were passing together through the gap region. This variation in trajectory shift is attributed to the particle-particle and particle-wall/droplet interactions. Such unavoidable interactions will certainly affect the separation efficiency of a concentrated particle mixture.

### Particle Size Effect

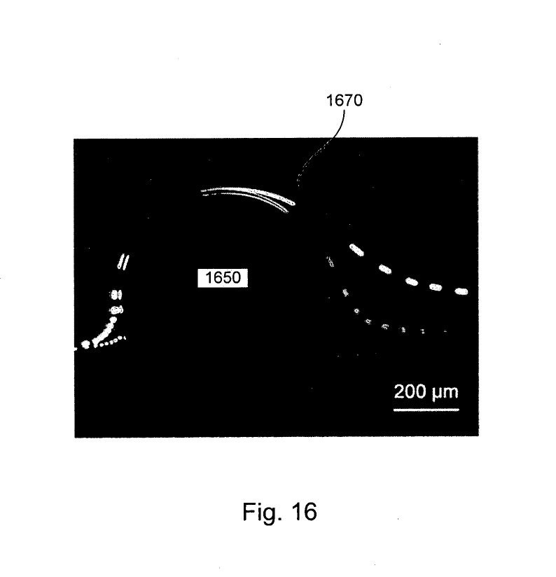

Based on the previous analysis, the optimal particle separation would occur when the gap is as small as possible, i.e. just large enough to allow continuous particle flow. In such a case, the highest possible field gradient and thus the highest DEP force would be achieved for the given voltages. Therefore, the gap was reduced to about 16 μm while the voltage at reservoir 1 was kept at 600 V. However, no particle separation or trajectory shift was observed due to a flow circulation region formed just before the gap. 15.7 μm particles seemed to be repulsed back from the gap region along with 5.7 μm particles and formed a so-called pearl chain, which is a known DEP phenomenon originated from particle-particle interaction. The chain circulated and trapped more incoming particles. The same phenomenon was observed when the voltage was reduced to 400 V; but after reducing it to 200 V, particles were able to flow through the gap and a 225 μm separation of the particle trajectories was observed. At the same conditions, we have also realized the successful separation of 5.7 μm particles from 1 mm particles as demonstrated in FIG. 16. It is noted that a 16× objective lens was used to visualize 1 μm particles in FIG. 16 while all previous figures were obtained with a 10× objective. As a side effect, the view field was limited to a narrow region around the droplet 1650 and did not cover the steady part of the 5.7 μm particles' trajectory. However, the trend of this trajectory is obvious and a 138 μm distance in the final y-coordinates for the two groups of particles can be assessed from FIG. 16.

FIG. 16 shows that the critical parameter in the DC-DEP particle separation is the gap size, i.e. the field gradient. For a successful separation the electrical field strength can have a very low value if the field gradient is sufficient to discriminate particles of different sizes. Choosing lower voltages for the DC-DEP separation is also preferable when dealing with biological samples since the biological cells are prone to lysis at a strong electrical field. High voltage also can cause significant Joule heating and gas bubble generation at metal electrodes due to the electrolysis. The only drawback of the low voltage separation is that the velocity of the particles and therefore separation process becomes slower.

In summary, the present invention, among other unique things, discloses a new particle or cell separation technology with high separation efficiency by a DC-DEP microfluidic device and method. Practicing this invention does not require any cell labeling and any complicated microfabrication of embedded micro-electrode arrays. This technology would provide a powerful tool for research labs and medical clinics to separate various blood cells by size. This technology will also overcome a major barrier for lab-on-chip technology: it will allow lab-on-chip devices to separate blood cells and other cells directly, so that a complete bio-medical analysis can be done on a single chip (i.e., the lab-on-chip devices no longer depend on using a centrifuge machine in conventional room-based laboratories for cell separation).

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

### LIST OF REFERENCES

(1) Rodriguez W R, Christodoulides N, Floriano P N, Graham S, Mohanty S, Dixon M, Hsiang M, Peter T, Zavahir S, Thior I, Romanovicz D, Bernard B, Goodey A P, Walker B D, McDevitt J T. A microchip CD4 counting method for HIV monitoring in resource-poor settings. PLoS Med. 2005; 2:e182.
(2) Taubenberger J K, Morens D M. 1918 influenza: the mother of all pandemics. Emerg Infect Dis. 2006; 12:15-22.
(3) UNAIDS. UNAIDS 2004 Report on the global AIDS epidemic. 1-20. 2004. UNAIDS.
(4) Moore R D, Chaisson R E. Natural history of opportunistic disease in an HIV-infected urban clinical cohort. Ann Intern Med. 1996; 124:633-42.
(5) Phair J, Munoz A, Detels R, Kaslow R, Rinaldo C, Saah A. The risk of Pneumocystis carinii pneumonia among men infected with human immunodeficiency virus type 1. Multicenter AIDS Cohort Study Group. N Engl J Med. 1990; 322:161-65.
(6) Bozzette S A, Finkelstein D M, Spector S A, Frame P, Powderly W G, He W, Phillips L, Craven D, van Der H C, Feinberg J. A randomized trial of three antipneumocystis agents in patients with advanced human immunodeficiency virus infection. NIAID AIDS Clinical Trials Group. N Engl J Med. 1995; 332:693-99.
(7) Masur H. Recommendations on prophylaxis and therapy for disseminated Mycobacterium avium complex disease in patients infected with the human immunodeficiency virus. Public Health Service Task Force on Prophylaxis and Therapy for Mycobacterium avium Complex. N Engl J Med. 1993; 329:898-904.
(8) Gordin F, Masur H. Prophylaxis of Mycobacterium avium complex bacteremia in patients with AIDS. Clin Infect Dis. 1994; 18 Suppl 3:S223-S226.
(9) Masur H, Kaplan J E, Holmes K K. Guidelines for preventing opportunistic infections among HIV-infected persons—2002. Recommendations of the U.S. Public Health Service and the Infectious Diseases Society of America. Ann Intern Med. 2002; 137:435-78.
(10) Corbett E L, Watt C J, Walker N, Maher D, Williams B G, Raviglione M C, Dye C. The growing burden of tuberculosis: global trends and interactions with the HIV epidemic. Arch Intern Med. 2003; 163:1009-21.
(11) Giorgi J V, Ho H N, Hirji K, Chou C C, Hultin L E, O'Rourke S, Park L, Margolick J B, Ferbas J, Phair J P. CD8+ lymphocyte activation at human immunodeficiency virus type I seroconversion: development of HLA-DR+ CD38− CD8+ cells is associated with subsequent stable CD4+ cell levels. The Multicenter AIDS Cohort Study Group. Journal of Infectious Diseases. 1994; 170:775-81.
(12) Giorgi J V, Liu Z, Hultin L E, Cumberland W G, Hennessey K, Detels R. Elevated levels of CD38+ CD8+ T cells in HIV infection add to the prognostic value of low CD4+ T cell levels: results of 6 years of follow-up. The Los Angeles Center, Multicenter AIDS Cohort Study. J Acquir Immune Defic Syndr. 1993; 6:904-12.
(13) Hulgan T, Raffanti S, Kheshti A, Blackwell R B, Rebeiro P F, Barkanic G, Ritz B, Sterling T R. CD4 lymphocyte percentage predicts disease progression in HIV-infected patients initiating highly active antiretroviral therapy with CD4 lymphocyte counts>350 lymphocytes/mm3. J Infect Dis. 2005; 192:950-957.
(14) Hammer S M. Clinical practice. Management of newly diagnosed HIV infection. N Engl J Med. 2005; 353:1702-10.
(15) Ahdieh-Grant L, Yamashita T E, Phair J P, Detels R, Wolinsky S M, Margolick J B, Rinaldo C R, Jacobson L P. When to initiate highly active antiretroviral therapy: a cohort approach. Am J Epidemiol. 2003; 157:738-46.
(16) Egger M, May M, Chene G, Phillips A N, Ledergerber B, Dabis F, Costagliola D, d'Arminio M A, de Wolf F, Reiss P, Lundgren J D, Justice A C, Staszewski S, Leport C, Hogg R S, Sabin C A, Gill M J, Salzberger B, Sterne J A. Prognosis of HIV-1-infected patients starting highly active antiretroviral therapy: a collaborative analysis of prospective studies. Lancet. 2002; 360:119-29.
(17) Hogg R S, Yip B, Chan K J, Wood E, Craib K J, O'Shaughnessy M V, Montaner J S. Rates of disease progression by baseline CD4 cell count and viral load after initiating triple-drug therapy. JAMA. 2001; 286:2568-77.
(18) Palella F J, Jr., Deloria-Knoll M, Chmiel J S, Moorman A C, Wood K C, Greenberg A E, Holmberg S D. Survival benefit of initiating antiretroviral therapy in HIV-infected persons in different CD4+ cell strata. Ann Intern Med. 2003; 138:620-626.
(19) Phillips A N, Staszewski S, Weber R, Kirk O, Francioli P, Miller V, Vernazza P, Lundgren J D, Ledergerber B. HIV viral load response to antiretroviral therapy according to the baseline CD4 cell count and viral load. JAMA. 2001; 286:2560-2567.
(20) Sterling T R, Chaisson R E, Keruly J, Moore R D. Improved outcomes with earlier initiation of highly active antiretroviral therapy among human immunodeficiency virus-infected patients who achieve durable virologic suppression: longer follow-up of an observational cohort study. J Infect Dis. 2003; 188:1659-65.
(21) Sterling T R, Chaisson R E, Moore R D. Initiation of highly active antiretroviral therapy at CD4+ T lymphocyte counts of >350 cells/mm3: disease progression, treatment durability, and drug toxicity. Clin Infect Dis. 2003; 36:812-15.
(22) Wood E, Hogg R S, Yip B, Harrigan P R, Montaner J S. Using baseline CD4 cell count and plasma HIV RNA to guide the initiation of highly active antiretroviral therapy. Rev Invest Clin. 2004; 56:232-36.
(23) Marquez, V. P., Sierra, V. H., Gayle, J., and Crown, R. HIV/AIDS in the Caribbean: Issues and Options. 1-65. 2000. Ref Type: Report
(24) Koenig S P, Leandre F, Farmer P E. Scaling-up HIV treatment programmes in resource-limited settings: the rural Haiti experience. Aids. 2004; 18 Suppl 3:S21-S25.
(25) Farmer P, Leandre F, Mukherjee J S, Claude M, Nevil P, Smith-Fawzi M C, Koenig S P, Castro A, Becerra M C, Sachs J, Attaran A, Kim J Y. Community-based approaches to HIV treatment in resource-poor settings. Lancet. 2001; 358:404-9.
(26) Peck R, Fitzgerald D W, Liautaud B, Deschamps M M, Verdier R I, Beaulieu M E, GrandPierre R, Joseph P, Severe P, Noel F, Wright P, Johnson Jr W D, Pape J W. The feasibility, demand, and effect of integrating primary care services with HIV voluntary counseling and testing: evaluation of a 15-year experience in Haiti, 1985-2000. J Acquir Immune Defic Syndr. 2003; 33:470-475.
(27) Severe P, Leger P, Charles M, Noel F, Bonhomme G, Bois G, George E, Kenel-Pierre S, Wright P F, Gulick R, Johnson W D, Jr., Pape J W, Fitzgerald D W. Antiretroviral therapy in a thousand patients with AIDS in Haiti. N Engl J Med. 2005; 353:2325-34.
(28) Crowe S, Turnbull S, Oelrichs R, Dunne A. Monitoring of human immunodeficiency virus infection in resource-constrained countries. Clin Infect Dis. 2003; 37:S25-S35.
(29) Kent D M, McGrath D, Ioannidis J P, Bennish M L. Suitable monitoring approaches to antiretroviral therapy in resource-poor settings: setting the research agenda. Clin Infect Dis. 2003; 37:S13-S24.
(30) Didier J M, Kazatchkine M D, Demouchy C, Moat C, Diagbouga S, Sepulveda C, Di Lonardo A M, Weiss L. Comparative assessment of five alternative methods for CD4+ T-lymphocyte enumeration for implementation in developing countries. J Acquir Immune Defic Syndr. 2001; 26:193-95.
(31) Landay A, Ho J L, Hom D, Russell T, Zwerner R, Minuty J G, Kataaha P, Mmiro F, Jackson B. A rapid manual method for CD4+ T-cell quantitation for use in developing countries. Aids. 1993; 7:1565-68.
(32) Malone J L, Simms T E, Gray G C, Wagner K F, Burge J R, Burke D S. Sources of variability in repeated T-helper lymphocyte counts from human immunodeficiency virus type 1-infected patients: total lymphocyte count fluctuations and diurnal cycle are important. J Acquir Immune Defic Syndr. 1990; 3:144-51.
(33) Schnizlein-Bick C T, Spritzler J, Wilkening C L, Nicholson J K A, O'Gorman M R G, Site I, The NIAID DAIDS New Technologies Evaluation Group. Evaluation of TruCount Absolute-Count Tubes for Determining CD4 and CD8 Cell Numbers in Human Immunodeficiency Virus-Positive Adults. Clin Diagn Lab Immunol. 2000; 7:336-43.
(34) Raboud J M, Haley L, Montaner J S, Murphy C, Januszewska M, Schechter M T. Quantification of the variation due to laboratory and physiologic sources in CD4 lymphocyte counts of clinically stable HIV-infected individuals. J Acquir Immune Defic Syndr Hum Retrovirol. 1995; 10 Suppl 2:S67-S73.
(35) Raboud J M, Montaner J S, Conway B, Haley L, Sherlock C, O'Shaughnessy M V, Schechter M T. Variation in plasma RNA levels, CD4 cell counts, and p24 antigen levels in clinically stable men with human immunodeficiency virus infection. J Infect Dis. 1996; 174:191-94.
(36) Sax P E, Boswell S L, White-Guthro M, Hirsch M S. Potential clinical implications of interlaboratory variability in CD4+ T-lymphocyte counts of patients infected with human immunodeficiency virus. Clin Infect Dis. 1995; 21:1121-25.
(37) Chabinyc M L, Chiu D T, McDonald J C, Stroock A D, Christian J F, Karger A M, Whitesides G M. An Integrated Fluorescence Detection System in Poly(dimethylsiloxane) for Microfluidic Applications. Anal Chem. 2001; 73:4491-98.
(38) Cui L, Zhang T, Morgan H. Optical particle detection integrated in a dielectrophoretic lab-on-a-chip. Journal of Micromechanics and Microengineering. 2002; 7-12.
(39) Fu L M, Yang R J, Lin C H, Pan Y J, Lee G B. Electrokinetically driven micro flow cytometers with integrated fiber optics for on-line cell/particle detection. Analytica Chimica Acta. 2004; 507:163-69.
(40) Lee G B, Lin C H, Chang G L. Micro flow cytometers with buried SU-8/SOG optical waveguides. Sensors and Actuators A: Physical. 2003; 103:165-70.
(41) Mogensen K B, El-Ali J, Wolff A, Kutter J P. Integration of polymer waveguides for optical detection in microfabricated chemical analysis systems. Appl Opt. 2003; 42:4072-79.
(42) Tung Y C, Zhang M, Lin C T, Kurabayashi K, Skerlos S J. PDMS-based opto-fluidic micro flow cytometer with two-color, multi-angle fluorescence detection capability using PIN photodiodes. Sensors and Actuators B: Chemical. 2004; 98:356-67.
(43) Xiang Q, Xuan X, Xu B., Li D. Multi-functional Particle Detection with Embedded Optical Fibers in a Poly(dimethylsiloxane) Chip. Instrumentation Sci & Tech. 2005; 33:597-607.
(44) Ren L, Li D. Theoretical studies of microfluidic dispensing processes. J Colloid Interface Sci. 2002; 254:384-95.
(45) Ren L, Sinton D, Li D. Numerical simulation of microfluidic injection processes in crossing microchannels. Journal of Micromechanics and Microengineering. 2003; 739-47.
(46) Sinton D, Ren L, Xuan X, Li D. Effects of liquid conductivity differences on multi-component sample injection, pumping and stacking in microfluidic chips. Lab Chip. 2003; 3:173-79.
(47) Sinton D, Ren L, Li D. A dynamic loading method for controlling on-chip microfluidic sample injection. J Colloid Interface Sci. 2003; 266:448-56.
(48) Sinton D, Ren L, Li D. Visualization and numerical modelling of microfluidic on-chip injection processes. J Colloid Interface Sci. 2003; 260:431-39.
(49) Pohl H A. Dielectrophoresis. London: Cambridge University Press; 1978.
(50) Lapizco-Encinas B H, Simmons B A, Cummings E B, Fintschenko Y. Insulator-based dielectrophoresis for the selective concentration and separation of live bacteria in water. Electrophoresis. 2004; 25:1695-704.
(51) Kang K H, Kang Y, Xuan X, Li D. Continuous separation of microparticles by size with Direct current-dielectrophoresis. Electrophoresis. 2006; 27:694-702.
(52) Duffy D C, Schueller O J A, Brittain S T, Whitesides G M. Rapid prototyping of microfluidic switches in poly(dimethyl siloxane) and their actuation by electro-osmotic flow. Journal of Micromechanics and Microengineering. 1999; 211-17.
(53) Xuan X, Xu B, Li D. Accelerated particle electrophoretic motion and separation in converging-diverging microchannels. Anal Chem. 2005; 77:4323-28.
(54) Kang K H, Li D. Dielectric Force and Relative Motion between Two Spherical Particles in Electrophoresis. Langmuir. 2006; 22:1602-8.
(55) McMullin J N, Qiao H, Goel S, Ren C L, Li D. Integrated optical measurement of microfluid velocity. Journal of Micromechanics and Microengineering. 2005; 1810-1816.
(56) Xiang Q, Hu G, Gao Y, Li D. Miniaturized immunoassay microfluidic system with electrokinetic control. Biosens Bioelectron. 2005.
(57) Xuan X, Raghibizadeh S, Li D. Wall effects on electrophoretic motion of spherical polystyrene particles in a rectangular poly(dimethylsiloxane) microchannel. J Colloid Interface Sci. 2005.
(58) Xuan X, Ye C, Li D. Near-wall electrophoretic motion of spherical particles in cylindrical capillaries. J Colloid Interface Sci. 2005; 289:286-90.
(59) Xuan X, Li D. Focused electrophoretic motion and selected electrokinetic dispensing of particles and cells in cross-microchannels. Electrophoresis. 2005; 26:3552-60.
(60) Ye C, Li D. Electrophoretic Motion of a Sphere in a Microchannel under the Gravitational Field. J Colloid Interface Sci. 2002; 251:331-38.
(61) Ye C, Sinton D, Erickson D, Li D. Electrophoretic Motion of a Circular Cylindrical Particle in a Circular Cylindrical Microchannel. Langmuir. 2002; 18:9095-101.
(62) Ye C, Li D. 3-D transient electrophoretic motion of a spherical particle in a T-shaped rectangular microchannel. J Colloid Interface Sci. 2004; 272:480-488.
(63) Ye C, Li D. Electrophoretic motion of two spherical particles in a rectangular microchannel. Microfluidics and Nanofluidics. 2004; 1:52-61.
(64) Ye C, Xuan X, Li D. Eccentric electrophoretic motion of a sphere in circular cylindrical microchannels. Microfluidics and Nanofluidics. 2005; 1:234-41.
(65) Young E W, Li D. Dielectrophoretic force on a sphere near a planar boundary. Langmuir. 2005; 21:12037-46.
(66) Steger, J. L., Dougherty, F. C., and Benek, J. A. A Chimera Grid Scheme. 1983. ASME Mini-Symposium on Advances in Grid Generation. Ref Type: Conference Proceeding
(67) Erickson D, Liu X, Krull U, Li D. Electrokinetically controlled DNA hybridization microfluidic chip enabling rapid target analysis. Anal Chem. 2004; 76:7269-77.
(68) Erickson D, Liu X, Venditti R, Li D, Krull U J. Electrokinetically based approach for single-nucleotide polymorphism discrimination using a microfluidic device. Anal Chem. 2005; 77:4000-4007.
(69) Gao Y, Hu G, Lin F Y, Sherman P M, Li D. An electrokinetically-controlled immunoassay for simultaneous detection of multiple microbial antigens. Biomed Microdevices. 2005; 7:301-12.
(70) Gao Y, Lin F Y H, Hu G, Sherman P M, Li D. Development of a novel electrokinetically driven microfluidic immunoassay for the detection of Helicobacter pylori. Analytica Chimica Acta. 2005; 543:109-16.
(71) Hu G, Gao Y, Sherman P, Li D. A microfluidic chip for heterogeneous immunoassay using electrokinetical control. Microfluidics and Nanofluidics. 2005; 1:346-55.
(72) Bland J M, Altman D G. Measuring agreement in method comparison studies. Stat Methods Med Res. 1999; 8:135-60.
(73) Cohen G B, Islam S A, Noble M S, Lau C, Brander C, Altfeld M A, Rosenberg E S, Schmitz J E, Cameron T O, Kalams S A. Clonotype Tracking of TCR Repertoires during Chronic Virus Infections. Virology. 2002; 304:474-84.
(74) Altfeld M A, Livingston B, Reshamwala N, Nguyen P T, Addo M M, Shea A, Newman M, Fikes J, Sidney J, Wentworth P, Chesnut R, Eldridge R L, Rosenberg E S, Robbins G K, Brander C, Sax P E, Boswell S, Flynn T, Buchbinder S, Goulder P J, Walker B D, Sette A, Kalams S A. Identification of novel HLA-A2-restricted human immunodeficiency virus type 1-specific cytotoxic T-lymphocyte epitopes predicted by the HLA-A2 supertype peptide-binding motif. Journal of Virology. 2001; 75:1301-11.
(75) Brander C, Hartman K E, Trocha A K, Jones N G, Johnson R P, Korber B, Wentworth P, Buchbinder S P, Wolinsky S, Walker B D, Kalams S A. Lack of strong immune selection pressure by the immunodominant, HLA-A*0201-restricted cytotoxic T lymphocyte response in chronic human immunodeficiency virus-1 infection. Journal of Clinical Investigation. 1998; 101:2559-66.
(76) Meyer-Olson D, Brady K W, Bartman M T, O'sullivan K M, Simons B C, Conrad J A, Duncan C B, Lorey S, Siddique A, Draenert R A, Addo M, Altfeld M, Rosenberg E, Allen T M, Walker B D, Kalams S A. Fluctuations of functionally distinct CD8+ T cell clonotypes demonstrate flexibility of the HIV-specific TCR repertoire. Blood. 2005.
(77) Meyer-Olson D, Brady K W, Blackard J T, Allen T M, Islam S, Shoukry N H, Hartman K, Walker C M, Kalams S A. Analysis of the TCR beta variable gene repertoire in chimpanzees: identification of functional homologs to human pseudogenes. J Immunol. 2003; 170:4161-69.
(78) Meyer-Olson D, Shoukry N H, Brady K W, Kim H, Olson D P, Hartman K, Shintani A K, Walker C M, Kalams S A. Limited T Cell Receptor Diversity of HCV-specific T Cell Responses Is Associated with CTL Escape. J Exp Med. 2004; 200:307-19.
(79) Anikeeva N, Lebedeva T, Krogsgaard M, Tetin S Y, Martinez-Hackert E, Kalams S A, Davis M M, Sykulev Y. Distinct molecular mechanisms account for the specificity of two different T-cell receptors. Biochemistry. 2003; 42:4709-16.
(80) Anikeeva N, Lebedeva T, Sumaroka M, Kalams S A, Sykulev Y. Soluble HIV-specific T cell receptor: expression, purification and analysis of the specificity. J Immunol Methods. 2003; 277:75-86.
(81) Lebedeva T, Anikeeva N, Kalams S A, Walker B D, Gaidarov I, Keen J H, Sykulev Y. Major histocompatibility complex class I-intercellular adhesion molecule-1 association on the surface of target cells: implications for antigen presentation to cytotoxic T lymphocytes. Immunology. 2004; 113:460-471.

What is claimed is:

1. A microchannel structure comprising:
a. a substrate having a first end, and an opposite, second end defining a body portion therebetween, wherein the body portion has a first surface and an opposite, second surface;
b. a first channel formed on the first surface of the substrate with a width, W₁, defined by a first sidewall and a second, opposite sidewall;
c. a second channel formed on the first surface of the substrate with a width, W₂, defined by a first sidewall and a second, opposite sidewall, wherein the second channel is in fluid communication with the first channel at a first at least three-way intersection; and
d. an insulating hurdle member having a top portion and protruding from the first sidewall of the first channel, wherein the top portion of the insulating hurdle member and the second sidewall of the first channel defines a width, W₁ₐ, therebetween, and wherein W₁ and W₁ₐ satisfy the relationship of W₁>W₁ₐ.

2. The microchannel structure of claim 1, wherein the top portion of the hurdle member has at least one corner with a corresponding angle α, wherein the angle α is in the range of 0 to 180°.

3. The microchannel structure of claim 2, wherein the hurdle member is substantially rectangular cross-sectionally, and the top portion of the hurdle member has two corners.

4. The microchannel structure of claim 2, wherein the hurdle member is substantially triangular cross-sectionally, and the top portion of the hurdle member has one corner.

5. The microchannel structure of claim 1, wherein the top portion of the hurdle member has a surface characterized by a curvature.

6. The microchannel structure of claim 5, wherein the hurdle member comprises an insulating liquid droplet, and the surface of the top portion of the hurdle member is at least partially spherical.

7. The microchannel structure of claim 1, further comprising a third channel formed on the first surface of the substrate with a width, W₃, defined by a first sidewall and a second, opposite sidewall, wherein the third channel is in fluid communication with the first channel at a second at least three-way intersection, wherein the third channel is formed on the first surface of the substrate such that the hurdle member is positioned between the first at least three-way intersection and the second at least three-way intersection.

8. The microchannel structure of claim 7, wherein the width, W₃, is same or different from at least one of the width, W₁, and the width, W₂.

9. The microchannel structure of claim 1, wherein the width, W₁, is same or different from width, W₂.

10. The microchannel structure of claim 1, wherein the substrate is formed with at least one insulating polymeric material.

11. The microchannel structure of claim 10, wherein the insulating polymeric material comprises PDMA.

12. The microchannel structure of claim 10, wherein the hurdle member is made from a material different from or substantially same as the at least one insulating polymeric material.

13. The microchannel structure of claim 1, wherein the first channel is formed with a first portion with a width, W₁, and a second portion with a width, W_{1b}, which is defined by a first sidewall portion and a second sidewall portion, wherein the first sidewall portion is located between the top portion of the hurdle member and the first at least three-way intersection, and the second sidewall portion is located between the top portion of the hurdle member and the first at least three-way intersection, respectively, and wherein the width, W_{1b}, is varied at least for a portion along the first channel between the top portion of the hurdle member and the first at least three-way intersection.

14. The microchannel structure of claim 13, wherein W_{1b}>W₁.

15. The microchannel structure of claim 14, wherein the width W_{1b} of the second portion of the first channel proximate to the first at least three-way intersection is larger than the width W_{1b} of the second portion of the first channel proximate to the hurdle member.

16. The microchannel structure of claim 1, further comprising an insulating base member, wherein the insulating base member is bonded with the substrate to form a sealed microchannel structure.

17. The microchannel structure of claim 16, wherein the insulating base member comprises a glass plate.

18. The microchannel structure of claim 1, wherein the first at least three-way intersection is substantially T-shaped.

19. The microchannel structure of claim 1, wherein the second at least three-way intersection is substantially T-shaped.

20. The microchannel structure of claim 7, further comprising a first well in fluid communication with the first channel at a first end of the first channel.

21. The microchannel structure of claim 20, further comprising a second well in fluid communication with the second channel at a first end of the second channel.

22. The microchannel structure of claim 21, further comprising a third well in fluid communication with the second channel at a second end of the second channel, which is apart from the first end of the second channel.

23. The microchannel structure of claim 22, further comprising a fourth well in fluid communication with the third channel at a first end of the third channel.

24. The microchannel structure of claim 23, further comprising a first electrode configured to be positioned in the first well and to be electrically connectable to a power source.

25. The microchannel structure of claim 24, further comprising a second electrode configured to be positioned in the second well and to be electrically connectable to a power source.

26. The microchannel structure of claim 25, further comprising a third electrode configured to be positioned in the third well and to be electrically connectable to a power source.

27. The microchannel structure of claim 26, further comprising a fourth electrode configured to be positioned in the fourth well and to be electrically connectable to a power source.

28. A microfluidic chip formed with one or more microchannel structures of claim 1.

29. A device made with one or more microfluidic chips of claim 28.

30. A method of separating particles or cells according to their sizes, wherein the size of each of the particles or cells is characterized by a corresponding diameter, comprising the steps of:
a. providing a microchannel structure having:
i. a substrate having a first end, and an opposite, second end defining a body portion therebetween, wherein the body portion has a first surface and an opposite, second surface;
ii. a first channel formed on the first surface of the substrate with a width, W₁, defined by a first sidewall and a second, opposite sidewall;
iii. a second channel formed on the first surface of the substrate with a width, W₂, defined by a first sidewall and a second, opposite sidewall, wherein the second channel is in fluid communication with the first channel at a first at least three-way intersection; and
iv. an insulating hurdle member having a top portion and protruding from the first sidewall of the first channel, wherein the top portion of the insulating hurdle member and the second sidewall of the first channel defines a width, W₁ₐ, therebetween, and wherein W₁ and W₁ₐ satisfy the relationship of W₁>W₁ₐ;
b. introducing a plurality of particles or cells in a liquid medium into the microchannel structure; and
c. applying a direct current (DC) electrical field within the microchannel structure to generate a non-uniform electrical field at least around the insulating hurdle member and a first voltage difference along the first channel such that the plurality of particles or cells is driven by the direct current (DC) electrical field along the first channel and separated according to their diameters by a dielectrophoretic force corresponding to the non-uniform electrical field when the plurality of particles or cells passes by the insulating hurdle member.

31. The method of claim 30, wherein the step of applying a direct current (DC) electrical field further comprising the step of generating a second voltage difference along the second channel such that at the first at least three-way intersection, a first group of the plurality of particles or cells moves to the second channel along a first direction, Y₁, and a second group of the plurality of particles or cells moves to the second channel along a second direction, Y₂, that is different from the first direction, respectively, wherein each of the first group of the plurality of particles or cells has a diameter that is larger than a predetermined diameter threshold, and each of the second group of the plurality of particles or cells has a diameter that is not larger than the predetermined diameter threshold.

32. The method of claim 30, further comprising the step of collecting particles or cells after the separation of particles or cells according to their sizes.

33. The method of claim 30, wherein the plurality of particles or cells comprises white blood cells and red blood cells.

34. The method of claim 30, wherein the plurality of particles or cells comprises normal cells and tumor cells.

35. The method of claim 30, wherein the microchannel structure further comprises a third channel formed on the first surface of the substrate with a width, W₃, defined by a first sidewall and a second, opposite sidewall, wherein the third channel is in fluid communication with the first channel at a second at least three-way intersection, wherein the third channel is formed on the first surface of the substrate such that the hurdle member is positioned between the first at least three-way intersection and the second at least three-way intersection.

36. A method of separating particles or cells according to their sizes, wherein the size of each of the particles or cells is characterized by a corresponding diameter, comprising the steps of:
a. providing a microchannel structure having at least one channel that is defined by a first sidewall and a second, opposite sidewall and has an insulating protrusion formed on one of the first sidewall and the second, opposite sidewall;
b. introducing a plurality of particles or cells in a liquid medium into the at least one channel; and
c. generating a non-uniform electrical field in the at least one channel such that when the plurality of particles or cells passes by the insulating protrusion, the plurality of particles or cells each receives a dielectrophoretic force proportional to its diameters, thereby being separable according to their sizes.

37. The method of claim 36, further comprising the step of collecting particles or cells after the separation of particles or cells.

38. The method of claim 36, wherein the plurality of particles or cells comprises white blood cells and red blood cells.

39. The method of claim 36, wherein the plurality of particles or cells comprises normal cells and tumor cells.

40. An apparatus of separating particles or cells according to their sizes, wherein the size of each of the particles or cells is characterized by a corresponding diameter, comprising:
a. a microchannel structure having at least one channel that is defined by a first sidewall and a second, opposite sidewall and has an insulating protrusion formed on one of the first sidewall and the second, opposite sidewall; and
b. means for generating a non-uniform electrical field in the at least one channel such that when the plurality of particles or cells in a liquid medium passes by the insulating protrusion, the plurality of particles or cells each receives a dielectrophoretic force proportional to its diameters, thereby being separable according to their sizes.

41. The apparatus of claim 40, wherein the means for generating a non-uniform electrical field comprises a DC power source.

42. The apparatus of claim 41, wherein the means for generating a non-uniform electrical field further comprises a plurality of electrodes configured to be electrically connectable to the DC power source and when connected to the DC power source, a non-uniform electrical field is generated at least in the at least one channel.

43. The apparatus of claim 42, wherein the means for generating a non-uniform electrical field further comprises a voltage controller electrically coupled to the DC power source and the plurality of electrodes, wherein the voltage controller is capable of controlling the voltage output of each of the plurality of electrodes individually.

44. The apparatus of claim 42, wherein the means for generating a non-uniform electrical field further comprises a conducting liquid medium containable in the at least one channel.

45. The apparatus of claim 40, further comprising means for receiving the plurality of particles or cells.

46. The apparatus of claim 40, further comprising means for collecting the plurality of particles or cells after the separation of the plurality of particles or cells.

47. The apparatus of claim 40, further comprising a plurality of microchannel structures in an array.
